(19) United States
(12) Patent Application Publication
(10) Pub. No.: US 2017/0231957 A1
(43) Pub. Date: Aug. 17, 2017

(54) INJECTABLE FORMULATIONS OF ASENAPINE

(71) Applicant: ALRISE Biosystems GmbH, Berlin (DE)

(72) Inventor: Celal ALBAYRAK, Berlin (DE)

(21) Appl. No.: 15/503,250

(22) PCT Filed: Jun. 11, 2015
(86) PCT No.: PCT/EP2015/063049

§ 371 Date: Feb. 10, 2017

(30) Foreign Application Priority Data
Aug. 14, 2014 (EP) 14180979.8

(51) Int. Cl. A61K 31/407; A61K 9/00; A61K 9/51

(52) U.S. Cl. CPC A61K 31/407; A61K 9/5153; A61K 9/0019

(57) ABSTRACT

The invention provides nano- and/or microparticles comprising asenapine or a pharmaceutically acceptable salt thereof, wherein said asenapine or said pharmaceutically acceptable salt thereof is embedded in a polymer matrix or encapsulated by a polymer shell, wherein the polymer matrix or the polymer shell comprises a polymer selected from polylactide, polyglycolide, and polyester copolymers comprising copolymerized units of lactic acid and/or glycolic acid, as well as a process for their production. Moreover, the invention provides pharmaceutical compositions comprising these particles.

The present invention relates to particle formulations containing asenapine as an active agent. In particular, formulations are provided which are suitable as injectable formulations for the sustained delivery of asenapine. Moreover, a process for the convenient and efficient production of such particle formulations is provided.

Asenapine has been approved by FDA (August 2009) in the form of sublingual tablets for the treatment of acute exacerbation of schizophrenia and acute manic or mixed episodes of bipolar disorder with or without psychosis in adults. To that extent, asenapine is one of the first psychotropic to obtain simultaneously FDA approval for schizophrenia and bipolar disorder.

Asenapine belongs to the so called “second generation antipsychotics”. The introduction of second generation antipsychotics (SGA) generated considerable optimism that better antipsychotic treatments for schizophrenia and bipolar disorder were possible. Second generation antipsychotics offer several tolerability benefits over first generation antipsychotics, particularly with respect to extrapyramidal symptoms. The unique receptor binding profile of asenapine shows promise in the treatment of positive and negative symptoms of schizophrenia with low risk of extrapyramidal and anticholinergic side effects. Based on its receptor pharmacology it is proposed that its efficacy is mediated by its antagonist activity on Dopamine (D)-2 and serotonin (5HT)-2A receptors. Asenapine had also been shown to have activities on HT1A, 5HT1B, 5HT6, 5HT7, D3 and alpha-2 adrenergic receptors which may be associated with improvements in cognition and negative and affective symptoms. Asenapine had been demonstrated to have the highest affinity for blocking serotonin receptors, followed by dopamine and alpha-adrenergic receptors with minimal affinity for muscarinic receptors (Australian Public Assessment Report for Asenapine, April 2011). Asenapine possesses a very favourable pharmacological profile regarding possible side effects (CNS Drugs 2012, 26(7), 601-611; Clinical Neuropharmacology 2013, 36(6), 223-238; Clinical Therapeutics, 2012, 34(5), 1023-1040).

Asenapine, formulations for asenapine and methods for treatment using asenapine are disclosed, e.g. in U.S. Pat. No. 4,145,434, EP 569 096 A1, WO 95/23600, WO 99/32108, US 2006/0084692, WO 2010/149727 or WO 2011/159903.

Schizophrenia is a serious chronic and devastating mental illness with a substantial impact on psychological, physical, social, and economical areas of an individual and society. It is recognized that a lifelong treatment with antipsychotics is necessary. For the treatment of such critical mental illness, a number of first generation (typical) and second generations (atypical) antipsychotics are currently available. Despite such treatment options, most of patients with schizophrenia had a poor treatment outcome.

The treatment with asenapine is impeded to some extent by non-optimal formulation paired with an unsuitable route for application. That remains the primary obstacle for the better treatment outcome in schizophrenia treatment with asenapine (Clinical Therapeutics 2012, 34(5), 1023). Commercially available asenapine formulations are used twice daily as sublingual tablets (bioavailability=35%), as disclosed in WO 95/23600. Because of a pronounced first pass effect of asenapine, oral formulations have not been successfully developed (bioavailability=2%) (J Pharmacology and Psychotherapeutics 2010, 1, 60). Depot preparations of asenapine in the form of a hemipamoate salt are disclosed in WO 2010/149727 A2.

Success of schizophrenia therapy and the treatment of other psychotic disorders depend critically on patient compliance and adherence. Generally, adherence to treatment in schizophrenia is regarded as central for optimizing recovery. However, non-adherence remains a significant clinical problem. And non-adherence to antipsychotic medication treatment leads to several clinical relevant outcomes, including relapse, increased clinical and emergency department visits, rehospitalisation and deterioration in social function.

US 2010/0130478 A1 discloses sustained release formulations, including microparticles, of psychoactive drugs in a matrix of tyrosine derived polyarylates. However, following the teaching of this document, particles with a high load of asenapine could not be obtained.

Microparticle depot formulations are available for risperidone as an alternative atypical antipsychotic drug. The depot formulation of risperidone is a two-week depot formulation marketed as Risperdal® Consta® by Janssen Cilag. After a single intramuscular (gluteal) injection of Risperdal® Consta®, there is a small initial release of the drug (<about 1% of the dose), followed by a lag time of 3 weeks. The main release of the drug starts from 3 weeks onward, is maintained from 4 to 6 weeks, and subsides by 7 weeks following the intramuscular (IM) injection. Due to the sigmoidal release profile of the two week depot formulation of risperidone, oral antipsychotic supplementation should be given during the first 3 weeks of treatment with Risperdal® Consta® to maintain therapeutic levels until the main release of risperidone from the injection site has begun. Moreover, in order to maintain therapeutic levels of risperidone in blood, Risperdal® Consta® has to be injected bi-weekly by a physician, i.e. frequent consultations are mandatory.

WO 2010/119455 A2 discloses the complexation of antipsychotic agents in the form of ion pair complexes with cholesteryl sulfate to provide injectable sustained release formulations.

Certain shortcomings, in particular the need for better patient compliance and adherence associated with the currently available formulations for asenapine, thus remain to be addressed despite the fact that asenapine offers a favourable pharmacological profile.

In order to facilitate the administration of asenapine, the present invention thus provides nano- and/or microparticles as defined in the annexed claims. The nano- and/or microparticles comprise asenapine or a pharmaceutically acceptable salt thereof embedded in a polymer matrix or encapsulated by a polymer shell, wherein the polymer matrix or the polymer shell comprises a polymer selected from polylactide, polyglycolide, and from polyester copolymers comprising copolymerized units of lactic acid and/or glycolic acid. Moreover, a process for the preparation of such nano- and/or microparticles is provided.

The nano- and/or microparticles in accordance with the invention can be conveniently administered to a patient via parenteral injection, in particular subcutaneous or intramuscular injection. Due to their composition, the particles provide an advantageous release profile. Surprisingly, the nano- and/or microparticles in accordance with the invention can be provided with a high load of the asenapin or its pharmaceutically acceptable salt, such that they can be used as depot formulation. Thus, the first pass effect can be avoided and the application frequency can be reduced. This, in turn, has a beneficial effect on patient compliance and adherence (Antipsychotic long acting injections, edited by P. Haddad, T. Lambert and J. Lauriello, Oxford University Press, 2011). The release rate of the active principle from the nano- and/or microparticles e.g. upon parenteral administration can be controlled so as to be able to respond to different pharmacological demands. Moreover, the particles show no, or no significant, initial burst in their release profile and no extended lag-phase. Rather, the active agent can be released in a continuous manner.

The process of the invention provides access to nano- and/or microparticles showing such a favourable release profile, and allows in addition the preparation of nano- and/or microparticles containing an amount of the drug which makes them particularly suitable as depot formulations.

The nano- and/or microparticles in accordance with the invention contain asenapine or a pharmaceutically acceptable salt thereof as an active principle. Herein, the asenapine free base and the pharmaceutically acceptable salts thereof, including preferred salts as disclosed below, may be collectively referred to as the active principle or active agent.

As will be understood by the skilled reader, asenapine is a non-proprietory name for the compound trans-5-chloro-2,3,3a,12b-tetrahydro-2-methyl-1-H-dibenz[2,3:6,7]oxepino[4,5-c]pyrrole or (3aRS,12bRS)-rel-5-chloro-2,3,3a,12b-tetrahydro-2-methyl-1H-dibenz[2,3:6,7]oxepino[4,5-c]pyrrole. It is generally used in the form of a racemic mixture of the (3aR,12bR)-form and the (3aS,12bS)-form.

Pharmaceutically acceptable salts of asenapine which can be used in the context of the present invention are in particular acid addition salts, which can be formed form the asenapine free base and an organic or inorganic acid. Exemplary acid addition salts can be selected from the group consisting of mineral acid salts such as hydrochloride, hydrobromide, hydroiodide, sulfate salts, nitrate salts, phosphate salts (such as, e.g., phosphate, hydrogenphosphate, or dihydrogenphosphate salts), carbonate salts, hydrogencarbonate salts or perchlorate salts; organic acid salts such as acetate, propionate, butyrate, pentanoate, hexanoate, heptanoate, octanoate, cyclopentanepropionate, undecanoate, lactate, maleate, oxalate, fumarate, tartrate, malate, citrate, nicotinate, benzoate, salicylate, pamoate or ascorbate salts; sulfonate salts such as methanesulfonate, ethanesulfonate, 2-hydroxyethanesulfonate, benzenesulfonate, p-toluenesulfonate (tosylate), 2-naphthalenesulfonate, 3-phenylsulfonate, or camphorsulfonate salts; and acidic amino acid salts such as aspartate or glutamate salts.

Particularly preferred as the active principle in the context of the present invention is the asenapine maleate salt.

As noted above, in accordance with the teaching provided herein, nano- and/or microparticles can be provided which show a continuous release of asenapine or a pharmaceutically acceptable salt thereof, preferably a continuous release of therapeutically active doses over extended periods of time. Surprisingly, advantageous release profiles can be achieved in the context of the invention without the need for any modification of the active principle, e.g. by complexation thereof.

The asenapine or the pharmaceutically acceptable salt thereof may be present in the nano- and/or microparticles in molecular dispersed form, amorphous or crystalline form, including a solvate or a hydrate form.

The content of the asenapine or the pharmaceutically acceptable salt thereof in the nano- and/or microparticles in accordance with the invention is typically at least 10 wt. %, based on the total weight of the nano- and/or microparticles. In this context, the content is expressed on the basis of the asenapine free base, e.g. in the case of a salt, the determined amount is recalculated as the corresponding amount of the free base. Preferably, the content of the asenapine or the pharmaceutically acceptable salt thereof is at least 15 wt %., and more preferably it is at least 20 wt %. Generally, the content of asenapine or the pharmaceutically acceptable salt thereof in the nano- and/or microparticles in accordance with the invention is not more than 50 wt. %, and more preferably not more than 40% wt. %, based on the total weight of the nano- and/or microparticles. In view of the favorably high drug load that can be provided in the nano- and/or microparticles in accordance with the invention, they can be suitably used as depot formulations for the active principle.

In the nano- and/or microparticles in accordance with the invention, the active principle is embedded in a polymer matrix or encapsulated by a polymer shell. The active principle is embedded or encapsulated in a solid form of the active principle, which may be a crystalline or amorphous form. Preferably, the active principle is embedded in a polymer matrix in the form of a solid dispersion of the active principle in the polymer matrix. This includes a crystalline dispersion (i.e. a form wherein the active principle forms crystalline phases dispersed in the polymer matrix), an amorphous dispersion (i.e. a form wherein the active principle forms amorphous phases in the polymer matrix), or a solid solution (i.e. a form where the active agent forms a molecular dispersion in the polymer matrix). In a solid dispersion of the active principle in the polymer matrix, the active principle may be dispersed across the full cross-section of the nano- and/or microparticles. However, for example if the nano- and/or microparticles carry a coating, there may be certain regions in the nano- and/or microparticles which remain free from the active principle.

In line with the above, in a preferred embodiment of the nano- and/or microparticles in accordance with the invention, these nano- and/or microparticles comprise asenapine maleate as an active agent dispersed in a polymer matrix, wherein the polymer matrix comprises a polymer selected from polylactide, polyglycolide, and polyester copolymers comprising copolymerized units of lactic acid and/or glycolic acid, and wherein the asenapin maleate is dispersed in the polymer matrix in an amount of at least 10 wt. %, in particular at least 15 wt. %, based on the total weight of the nano- and/or microparticles.

The polymer forming the polymer matrix or the polymer shell comprises a polymer selected from polylactide, polyglycolide, and polyester copolymers comprising copolymerized units of lactic acid and/or glycolic acid.

Such homopolymers and copolymers are known to the skilled person and established for use in the medical field (e.g. Biodegradable polymers in clinical use and clinical Development. Edited by A. Domb, N. Kumar and A. Ezra, Wiley, 2011, Long Acting Injections and Implants Editors: J. C. Wright and D. J. Burgess, Springer 2012). They are typically biodegradable. Suitable polymers include in particular polyglycolide homopolymers (PGA, also referred to as polyglycolic acid), and polylactide homopolymers (PLA, also referred to as polylactic acid). Since polymers formed from lactic acid as monomer units can contain the units in D- or L-configuration, lactic acid may form a homopolymer containing only one of the two enantiomers (e.g. poly(L-lactic acid), PLLA), or a polymer combining units of L- and D-lactic acid. The latter are also referred to as stereo-copolymers. The stereo-copolymers may have different arrangements of the comonomers, and form e.g. random or block copolymers (e.g. poly(DL-lactic acid) random copolymer, or L-lactic acid/DL-lactic acid copolymers. Such stereo-copolymers formed from lactic acid as monomer unit are also suitable for use in the context of the invention. Unless indicated otherwise in a specific context, reference to polymerized lactide or lactic acid units includes L-lactic acid units, D-lactic acid units, or combinations of the two. Moreover, unless indicated otherwise in a specific context, reference to polylactide homopolymers includes not only polymers consisting of D-lactic acid or L-lactic acid units, but also polymers combining D-lactic acid and L-lactic acid units.

As particularly suitable polyester copolymers comprising copolymerized units of lactic acid and/or glycolic acid, reference can be made to a copolymer of lactide and glycolide, i.e. poly(lactide-co-glycolide), also referred to as poly(lactic-co-glycolic acid), PLGA. As will be appreciated by the skilled person, the degradation rate of such polymers after administration can be controlled by the ratio of copolymerized units of lactic acid to glycolic acid in the copolymer. Among these poly(lactide-co-glycolide) copolymers, preferred are those wherein the content of polymerized lactic acid units is at least 50 mol %, and in particular those wherein the content of polymerized lactic acid units is 50 to 85 mol %, such as 50 or 75 mol %, based on the total amount of polymerized units. As examples of other suitable comonomers that may be present in polyester copolymers comprising copolymerized units of lactic acid and/or glycolic acid, one or more comonomers selected from tetramethylglycolide, δ-valerolactone, ε-caprolactone, trimethylene carbonate, tetramethylglycolide, and ethylene glycol may be mentioned. Thus, exemplary polyester copolymers, preferably binary copolymers, comprising copolymerized units of lactic acid and/or glycolic acid include a copolymer selected from the group consisting of a copolymer of glycolide and tetramethylglycolide, a copolymer of glycolide and δ-valerolactone, a copolymer of glycolide and ε-caprolactone, a copolymer of glycolide and trimethylene carbonate, a copolymer of lactide and tetramethylglycolide, a copolymer of lactide and δ-valerolactone, a copolymer of lactide and ε-caprolactone, a copolymer of lactide and trimethylene carbonate, a copolymer of glycolide and ethylene glycol, and a copolymer of lactide and ethylene glycol.

The polyester copolymers include random copolymers, block copolymers and gradient copolymers. Suitable block copolymer architectures include, e.g. AB block copolymers (e.g. AB block polymers comprising a polylactide (PLA) block and a poly(ethylene glycol) (PEG) block), ABA tri-block copolymers (e.g. ABA tri-block copolymers comprising PLA-PEG-PLA), star-shaped block copolymers (e.g. S(3)-PEG-PLA block copolymers and S(4)-PEG-PLA block copolymers).

In the polyester copolymers comprising copolymerized units of lactic acid and/or glycolic acid for use in the context of the present invention, it is preferred that the amount of the copolymerized units of lactic acid and/or glycolic acid (i.e. the amount of copolymerized units of lactic acid, if no glycolic acid is copolymerized, the amount of copolymerized units of glycolic acid, if no lactic acid is copolymerized, or the sum of the amounts of copolymerized units of glycolic acid and lactic acid, if both are copolymerized) accounts for at least 50 mol % of all copolymerized units in the copolymer. It is more preferred that that the amount of the copolymerized units of lactic acid and/or glycolic acid accounts for at least 70 mol % of all copolymerized units in the copolymer.

It will be appreciated by the skilled reader that the degradation rate of the nano- and/or microparticles of the invention can be influenced by the molecular weight of the polymer. Polymers of different molecular weights (or inherent viscosities) can be mixed to yield a desired degradation profile. Generally, polymers with an intrinsic viscosity in the range of 0.1 to 3 dL/g, preferably 0.1 to 2 dL/g (0.1% (w/v), chloroform, at 25° C.) are used.

Particularly preferred for use in the context of the present invention are nano- and/or microparticles comprising a poly(lactide-co-glycolide) copolymer, i.e. a copolymer consisting of glycolic acid and lactic acid units. In terms of their relative amount of glycolic acid and lactic acid units, preferred are those wherein the content of polymerized lactic acid units is at least 50 mol %, and in particular those wherein the content of polymerized lactic acid units is 50 to 85 mol %, such as 50 or 75 mol %, based on the total amount of polymerized units. Blends of poly(lactide-co-glycolide) copolymers with different relative amounts of glycolic acid and lactic acid, favourably within the above preferred limits, may also be used.

Suitable commercially obtainable polymers for use in the nano- and/or microparticles according to the present invention include, but are not limited to Resomer® (EVONIK) L-104, L-206, L-207, L-208, L-209, L-210, L-214, R-104, R-202, R-203, R-206, R-207, R-208, G-110, G-205, LR-909, RG-502, RG-502H, RG-503, RG-503H, RG-504, RG 504H, RG-505, RG-505H, RG-506, RG-508, RG-752, RG-752H, RG-753, RG753H, RG-755, RG-756, RG-757 and Resomer® RG-858.

The polymer matrix or polymer shell of the nano- and/or microparticles may comprise one type of polymer selected from polylactide, polyglycolide, and polyester copolymers comprising copolymerized units of lactic acid and/or glycolic acid, or two or more types of polymers selected from polylactide, polyglycolide, and polyester copolymers comprising copolymerized units of lactic acid and/or glycolic acid, e.g. as a polymer blend. If two or more types are used, the polymers may differ in the type of polymerized monomer units, or in the relative ratios thereof. It is preferred that the one or more polymers selected from polylactide, polyglycolide, and polyester copolymers comprising copolymerized units of lactic acid and/or glycolic acid account for 70 wt % or more, in particular 80 wt % or more of the polymers in the polymer matrix or polymer shell of the nano- and/or microparticles. It is particularly preferred that the polymer matrix or polymer shell does not contain any other polymer component, apart from the one or more polymers selected from polylactide, polyglycolide, and polyester copolymers comprising copolymerized units of lactic acid and/or glycolic acid.

In line with the above, in a particularly preferred embodiment of the nano- and/or microparticles in accordance with the invention, these nano- and/or microparticles comprise asenapine maleate as an active agent dispersed in a polymer matrix, wherein the polymer matrix comprises a poly(lactide-co-glycolide) copolymer, and wherein the asenapin maleate is dispersed in the polymer matrix in an amount of at least 10 wt. %, in particular at least 15 wt. %, based on the total weight of the nano- and/or microparticles.

As will be understood by the skilled reader, the reference to “nano- and/or microparticles” indicates that the particles may be completely or predominantly in the nanometer size range (such as 10 to 100 nm), that they may be completely or predominantly in the micrometer size range (such as >0.1 to 1000 μm, or preferably >0.1 to 100 μm), or that particle mixtures of nano- and microparticles can be prepared and can be used in the context of the invention. Typically, the particle size of the nano- and/or microparticles in the context of the present invention, as determined e.g. by laser scattering, ranges from 10 nm to 1000 μm, preferably from 50 nm to 300 μm. The d₉₀ value, determined via laser scattering on a particle number basis, is preferably below 100 μm, more preferably below 50 μm. The mean particle diameter based on a particle volume basis generally ranges from 10 nm to 200 μm, preferably from 400 nm to 150 μm, more preferably from 1 μm to 125 μm, and in particular from 5 μm to 125 μm. The mean diameter is determined by laser scattering and calculated as volume weighted mean diameter that represents the arithmetic mean size in volume %, mode (D(4,3)). As explained below, a desired particle size can be obtained by suitably choosing the process parameters in the production of the nano- and/or microparticles. Moreover, it will be understood that the particle size distribution, or the upper or the lower limit of the particle size, may be adjusted, if necessary, via conventional methods such as sieving or other forms of powder classification.

It is an advantage of the nano- and/or microparticles obtainable by the process in accordance with the invention that they are generally non-agglomerating. Preferably, the nano- and/or microparticles are nano- and/or microspheres.

The nano- and/or microparticles in accordance with the invention comprise asenapine or a pharmaceutically acceptable salt thereof embedded in a polymer matrix or encapsulated by a polymer shell, wherein the polymer matrix or the polymer shell comprises a polymer selected from polylactide, polyglycolide, and polyester copolymers comprising copolymerized units of lactic acid and/or glycolic acid. Optionally, they may comprise in addition excipients, including, e.g., one or more selected from a colorant, a vehicle, a preservative, an antioxidant, a buffer, a surfactant, and a flavoring agent, or a further active agent to be co-administered with the asenapine or its pharmaceutically active salt. For example, optional additional components may be admixed within the particles, or coated onto the particles. It is preferred that the asenapine or the pharmaceutically acceptable salt thereof, and the polymer selected from polylactide, polyglycolide, and polyester copolymers comprising copolymerized units of lactic acid and/or glycolic acid account for 80 wt. % or more, in particular 90 wt. % or more of the total weight of the nano- and/or microparticles. It is particularly preferred that the nano- and/or microparticles consist of (i) the asenapine or the pharmaceutically acceptable salt thereof, (ii) the polymer selected from polylactide, polyglycolide, and polyester copolymers comprising copolymerized units of lactic acid and/or glycolic acid, and (iii) optionally up to a maximum of 10 wt %, preferably up to a maximum of 5 wt %, based on the total weight of the nano- and/or microparticles, of one or more of a surfactant, another suitable excipient, and residual solvent.

It is preferred that agents intended for the complexation of the asenapine are not contained in the nano- and/or microparticles, such as cholesterylsulfate or its alkali metal salt.

Asenapine and pharmaceutically acceptable salts thereof are commercially available, or may be prepared in accordance with synthetic methods as disclosed, e.g., in U.S. Pat. No. 4,145,434, US 2006/0229352 A1, WO 2011/159903 A2 or the references cited therein.

The nano- and/or microparticles comprising asenapine or a pharmaceutically acceptable salt thereof in accordance with the invention can be advantageously prepared by a process comprising the steps of:
a) providing a solution of a polymer in an organic solvent S1 having limited water solubility; wherein said polymer is selected from the group consisting of polylactide, polyglycolide, and polyester copolymers comprising copolymerized units of lactic acid and/or glycolic acid;
b) combining the solution provided in step a) with asenapine or a pharmaceutically acceptable salt thereof by
   b1) dispersing the asenapine or the pharmaceutically acceptable salt thereof in the solution provided in step a), or
   b2) providing a solution or dispersion of the asenapine or the pharmaceutically acceptable salt thereof in an organic solvent S2 and combining the solution or dispersion with the solution provided in step a),
to provide an organic phase which comprises dissolved polymer and asenapine or a pharmaceutically acceptable salt thereof dissolved or dispersed therein;
c) agitating the organic phase provided in step b) in a vessel and adding an aqueous surfactant solution to the agitated organic phase in a volume ratio of at least 2:1 in terms of the total volume of the aqueous surfactant solution to the total volume of the organic phase provided in step b), thus causing the formation of a dispersion containing a continuous aqueous phase and a discontinuous organic phase; and
d) allowing the formation of a suspension of the nano- and/or microparticles via transfer of organic solvent from the discontinuous organic phase into the aqueous surfactant phase directly after the dispersion has been formed.

Surprisingly, it was found that the formation of nano- and/or microparticles relying on this process allows advantageously high loads of asenapine or a pharmaceutically acceptable salt thereof to be obtained, e.g. compared to other methods established for the formation of drug loaded particles. Reference is made in this respect to the particles resulting from the double emulsion method used in the comparative examples 1 and 2 contained herein. Moreover, the process allows nano- and/or microparticlces to be obtained with a favourable release profile of the active principle. The process according to the present invention provides convenient control of the particle size and the particle size distribution. It can be carried out as a simple one-pot process and a may readily be scaled up to meet commercial-scale production needs. Moreover, it is very efficient in that it enables a reduction of the energy and the time required for the production of the particles. In addition, comparably small amounts of solvents and surfactants as well as toxicologically acceptable solvents can be conveniently used. The encapsulation efficiency (i.e. the ratio of the active principle incorporated into the nano- and/or microparticles, including both the case where the active principle is embedded in a polymer matrix and where the active principle is encapsulated by a polymer shell) is high, and typically 70% (wt/wt) or more, preferably 75% or more, and more preferably 80% or more, in terms of the ratio of the actual content of the active principle in the nano- and/or microparticles, divided by the theoretical content×100.

In order to provide the solution of the polymer in step a), the polymer selected from polylactide, polyglycolide, and polyester copolymers comprising copolymerized units of lactic acid and/or glycolic acid is dissolved in an organic solvent (S1) which is a solvent for the polymer and has a limited water solubility. Preferably, the solubility of S1 in water is 1 to 60 wt % (or 10 to 600 g/L) at 20° C. (as wt % or weight of the solvent S1 in relation to the total weight of the mixed phase containing water and the solvent S1), more preferably 2 to 40 wt % (20 to 400 g/L), and in particular 4 to 40 wt % (40 to 400 g/L).

The solubilities of the polymers suitable for use in the context of the present invention in numerous organic solvents are reported in the literature or can be tested in a straightforward manner. The same applies for the solubility of solvents in water, for which values can be derived from numerous standard collections of physical and chemical data, such as the CRC Handbook of Chemistry and Physics, Taylor & Francis. The following table provides an additional overview.

| | boiling point* | solubility in water** |
|---|---|---|
| | [° C.] | [g/L] (20° C.) |
| ethyl formate | 54.5 | 105*** |
| ethyl acetate | 77.06 | 85.3 |
| methyl acetate | 57 | 319 |
| methyl formate | −31.5 | 300 |
| acetone | 56.2 | soluble |
| butyl acetate | 126.5 | 7 |
| n-propyl acetate | 101.6 | 21.2 |
| isopropyl acetate | 90 | 31 |
| n-propyl formate | 81.3 | 28 |
| DMSO | 189 | 1000 |
| NMP | 202 | 1000 |
| THF | 67 | soluble |
| glycofurol | 328 | soluble |
| methylisopropylketone | 94-95 | 6 |
| ethylmethylketone (EMK) | 79.6 | 292 |
| dimethyl carbonate | 90-91 | 139 |

*Handbook of Chemistry and Physics, CRC Press, 65^{th} edition, 1984-1985
**Merck Chemicals Product Information
***Gestis Stoffdatenbank

Preferably, the polymer should be soluble in the solvent S1 in an amount of 100 g/L, or more at 20° C. as the weight of the dissolved substance (the solute) per volume of the solvent (i.e. the volume of solvent added to the solute).

Preferred solvents S1 are selected from alkyl acetates, especially C1-C3 alkyl acetates, alkyl formates, especially C1-C3 alkyl formates, and mixtures of two or more thereof. Particularly preferred are solvents S1 selected from ethyl acetate, methyl acetate, ethyl formate, propyl formate, and mixtures of two or more thereof.

The asenapine or a pharmaceutically acceptable salt thereof is dissolved in the organic phase or dispersed as a solid in the organic phase. In order to effectively dissolve the asenapine or a pharmaceutically acceptable salt thereof in the organic phase, a solution of the active principle in a solvent S2 can be provided, and this solution can be combined with the solution of the polymer in the organic solvent S1. It will be understood that, in this case, the solvents S1 and S2 should be different solvents. It is also possible to add the active principle to the solvent S2 in an amount that only a part of it is dissolved in S2, and a part of it remains dispersed as a solid in the solvent S2. Using the process described herein, the active principle can also be encapsulated/embedded in the polymer at a high efficiency in this case. Preferably, the solubility of the active principle in the organic solvent S2 should be 10 g/L or higher, more preferably 100 g/L or higher at 20° C. In this context, the solubility is indicated as the weight of the dissolved substance (the solute) per volume of the solvent (i.e. the volume of solvent added to the solute). The solubilities of asenapine and its salts in diverse solvents are reported in the literature or can be tested in a straightforward manner. Nevertheless, the following table summarizes the solubility of asenapine maleate in a variety of solvents.

| Solvent | Concentration [mg/mL] |
|---|---|
| DMSO | 700 |
| NMP | 700 |
| benzyl alcohol | 500 |
| ethyl acetate | 10 |
| ethyl formate | 100 |
| methyl acetate | 25 |
| ethanol | 100 |
| isopropanol | 7 |
| glycofurol | 270 |
| EMK | 50 |
| acetone | 67 |
| THF | 100 |
| dimethycarbonate | 7 |
| dimethylacetamide | 500 |
| isopropyl acetate | 9 |
| isopropyl formate | 100 |
| benzyl benzoate | 7 |
| ethyl benzoate | 10 |
| methyl benzoate | 20 |

The organic solvent S2 should preferably be able to act also as a solvent for the polymer selected from polylactide, polyglycolide, and polyester copolymers comprising copolymerized units of lactic acid and/or glycolic acid. Preferably, the solubility of the polymer in the mixture of solvents S1+S2 is 100 g/L or higher at 20° C. as the weight of the dissolved substance (the solute) per volume of the solvent (i.e. the volume of solvent added to the solute).

A preferred organic solvent S2 is selected from alkyl esters of benzoic acid, in particular C1-C3 alkyl esters of benzoic acid, aryl esters of benzoic acid, in particular benzoic acid phenyl ester, benzyl alcohol, dimethyl sulfoxide (DMSO), N-methyl pyrrolidone (NMP), glycofurol and mixtures thereof. Particularly preferred as solvent S2 are benzyl alcohol, DMSO, NMP, and glycofurol.

If a solvent S2 is used, the volume ratio of solvent S2 to solvent S1 which are combined to provide the organic phase in step b) of the process of the invention, is preferably 5-50 vol % S2 to 50-95 vol % S1, based on the sum of the volumes S1+S2 prior to their combination as 100 vol %. Particularly preferred are ratios of 20-50 vol % S2 to 50-80 vol % S1. It will be understood that the solvent S2 should preferably be miscible with the solvent S1 at the ratios in which these solvents are used.

In order to disperse the asenapine or a pharmaceutically acceptable salt thereof in the organic phase in accordance with the alternative embodiment b1) of the process in accordance with the invention, the active principle can be added to the organic phase and mechanically dispersed using standard equipment.

The organic phase provided in step b) of the process in accordance with the invention contains an organic solvent S1 and optionally an organic solvent S2. Unless indicated otherwise, or dictated by a specific context, any reference to solvent S1 or solvent S2 is intended to include the option that more than one of S1 or S2, respectively, is used. Further solvents or water may be present in the organic phase in addition to S1 and optionally S2, as far as they do not have a negative impact on the process. However, it is generally preferred that the solvent or solvent mixture used to provide the organic phase in step b) of the process in accordance with the invention contains at least 80, more preferably at least 90% (vol./vol., based on the total volume of solvents in the organic phase) of S1 and optionally S2, and it is most preferred that the solvent or solvent mixture consists of S1 and optionally S2. Moreover, it is generally preferred that no halogenated solvents are used in the process of the invention as solvent S1, S2 or as an additional solvent.

In the organic phase provided in step b) of the process in accordance with the invention, the content of the dissolved polymer selected from polylactide, polyglycolide, and polyester copolymers comprising copolymerized units of lactic acid and/or glycolic acid is preferably 1 wt % to 40 wt %, based on the total weight of the organic phase. More preferably, the organic phase contains 5 to 40 wt % of the dissolved polymer. The concentration of the active principle can be suitably chosen with a view to the desired drug load of the resulting particles discussed above. The process of the present invention is capable of incorporating the active principle into the nano- and/or microparticles with a high efficiency, such that a high ratio of typically 70% or more, preferably 75% or more, and in particular 80% (wt/wt) or more of the active principle dissolved or dispersed in the organic phase b) will be incorporated into the particles. For example, asenapine maleate in a concentration of 5 to 30 wt %, based on the total weight of the organic phase, can be suitably used.

After the organic phase containing the polymer and the active principle has been provided in step b), an aqueous surfactant solution is added thereto. The addition of the aqueous surfactant solution to the organic phase provided in step b) is carried out while the organic phase provided in step b) is agitated, e.g. stirred. Preferably, the organic phase is stirred in a vessel while the aqueous surfactant solution is added. Typically, the aqueous surfactant solution is added in a continuous manner, or in multiple steps. Preferably, the surfactant solution is added to the total volume of the organic phase such that the content of the surfactant solution in the combination of the surfactant solution and organic phase gradually increases until the addition is completed. The addition may take place, e.g., over a time period of 5 s to 5 min, preferably 10 s to 2 min. Thus, a generally preferred form of the addition of the aqueous surfactant solution in step c) to the organic phase provided in step b) is to add the aqueous surfactant solution to the total volume of the organic phase under stirring such that the content of the surfactant solution in the combined surfactant solution and organic phase gradually increases until the addition is completed, and the addition takes place over a period of time of 5 s to 5 min, more preferably 10 s to 2 min. For example, the surfactant solution can be poured into the stirred organic phase over a time period of 5 s to 5 min, preferably 10 s to 2 min.

It is preferred for reasons of efficiency to provide the organic phase in step b) in a vessel with a volume that is sufficiently large to additionally accommodate the volume of the surfactant solution to be added. In this case, the process can be carried out as a one-pot process, i.e. a process where the organic phase containing the active principle and the polymer, and the desired nano- and/or microparticles can be prepared in a single vessel in subsequent steps. Thus, steps b), c) and d) can take place in the same vessel.

The aqueous surfactant solution added in step c) contains water, optionally in combination with one or more organic solvents. However, as implied by the term “aqueous”, water is the main solvent in the aqueous surfactant solution with a volume ratio of more than 50 vol % of the total volume of the solvents combined to form the aqueous surfactant solution, preferably more than 70 vol %, and more preferably more than 90 vol %. Preferably, the solvent(s) in the aqueous surfactant solution consist(s) of water, or of water in combination with a co-solvent which is fully miscible in all proportions with water. As an optional co-solvent to assist in the extraction of the solvent(s) from the organic phase, an alcohol, in particular a C1-C4 alcohol, and preferably a C1-C3 alcohol, such as ethanol may be mentioned. Alternatively, water may be the only solvent in the aqueous surfactant solution. Reference temperature for the volume ratio is 20° C.

The concentration of the surfactant in the aqueous surfactant solution is preferably in the range of 0.1% (w/v) to 30% (w/v), preferably 0.1% to 20%, and more preferably 0.1 to 5%, based on the total volume of the surfactant solution. As will be understood by the skilled reader, the concentration in weight per volume corresponds to the amount of the solute in g per 100 ml of the total volume of the solution including the surfactant, typically at 20° C.

Surfactants suitable for the aqueous surfactant solution encompass cationic-, anionic-, and non-ionic surfactants. Exemplary surfactants can be selected from polyoxyethylene-polyoxypropylene block copolymers, in particular polyoxyethylene-polyoxypropylene-polyoxyethylene-triblock copolymers such as Poloxamer®, Poloxamine®, polyethylene glycol alkyl ethers, fatty acid esters of polyoxyethylensorbitan, especially polyoxyethylenesorbitan monooleate and polyoxyethylenesorbitan monolaurate also referred to as polysorbates (Tween®, Span®), sucrose esters (Sisterna®, Ryoto Sugar Ester, Tokyo), gelatin, polyvinylpyrrolidone, fatty alcohol polyglycosides, Charps, Charpso, decyl-β-D-glycopyranoside, decyl-β-D-maltopyranoside, dodecyl-β-D-maltopyranoside, sodium oleate, polyethylene glycol, polyvinyl alcohol (PVA), polyethoxylated fatty acid ethers (Brij®), Triton X 100 or mixtures thereof. Preferred as a surfactant are polyvinyl alcohol, polyoxyethylene-polyoxypropylene-polyoxyethylene-triblock copolymers and fatty acid esters of polyoxyethylensorbitan, especially polyoxyethylenesorbitan monooleate and polyoxyethylenesorbitan monolaurate.

The aqueous surfactant solution may contain other components besides the water, optional additional solvents and the surfactant, e.g. a buffer, an agent for adjusting the viscosity of the aqueous surfactant solution, or an agent for adjusting the ion strength of the solution. For example, the aqueous surfactant solution may comprise a dissolved salt, such as NaCl, or dissolved sugar.

In the process according to the invention, the aqueous surfactant solution is added in step c) to the organic phase provided in step b) in a volume ratio of at least 2:1 in terms of the total volume of the aqueous surfactant solution to the total volume of the organic phase provided in step b) and prior to the addition of the aqueous surfactant solution. Preferably, the volume ratio is at least 3:1. While it is possible to use very large volumes of the aqueous surfactant solution to prepare the nano- or microparticles, it is preferred to keep the volume at a low level in order to reduce the consumption of solvents and other components. Thus, the volume ratio of the total volume of the aqueous surfactant solution to the total volume of the organic phase is generally not more than 10:1, preferably not more than 5:1. The volume of the aqueous surfactant solution is typically sufficiently large such that it can dissolve at least the solvent S1 contained in the organic phase to which the aqueous surfactant solution is added.

The addition of the aqueous surfactant solution to the organic phase at a volume ratio as defined above causes the formation of a dispersion containing a continuous aqueous phase and a discontinuous organic phase. Due to the water solubility at least of the solvent S1, the aqueous surfactant solution not only forms the continuous phase in the resulting dispersion, but acts at the same time as an extraction medium for the solvent S1 wherein the polymer had been dissolved. Thus, the solvent S1 is transferred from the organic phase to the aqueous continuous phase. If solvent S2 is used, it may be transferred fully or in part, depending on its water solubility to the aqueous continuous phase. This process may proceed via an emulsion of the organic phase as a discontinuous phase in the aqueous surfactant phase as an intermediate. However, since the solvent S1 is soluble in water to a certain extent, S1 is extracted from the organic phase into the continuous phase thereby leading to the formation of solid nano- and micro particles. Thus, a stable emulsion can typically not be observed in the process. Rather, once a continuous aqueous phase has been formed in step c) of the process in accordance with the invention, a suspension of the nano- and/or microparticles is directly formed in step d). A certain amount of solvent S1 and/or S2 may remain in the nano- and/or microparticles, and can be removed in (optional) subsequent extraction steps.

The transfer of at least the organic solvent S1 occurs in step d) from the organic phase to the aqueous surfactant phase, generally via diffusion of the organic solvent into the aqueous phase, and via dissolution of the organic solvent in the aqueous phase. The polymer and the active principle are left in the discontinuous organic phase, and a suspension of nano- and/or microparticles is formed in this manner. The formation of the suspension of nano- and/or microparticles takes place within minutes, or even less than a minute, after the start of the addition of the aqueous surfactant solution in step c). Typically, nano- and/or microparticles can be observed immediately after the formation of a continuous aqueous phase in step c). The formation of the particles takes place spontaneously when the aqueous surfactant solution is added in step c), i.e. without the need for any further activity triggering the formation, such as the removal of a solvent from the mixture e.g. via evaporation. However, further steps such as the extraction of solvent S2 remaining in the nano- and/or microparticles, e.g. with a mixture of water and a co-solvent, or the removal of organic solvent(s) from the system during or after the formation of the suspension of nano- and/or microparticles, can be optionally added to the process in accordance with the invention. The organic solvent(s) can be removed, e.g., via evaporation or extraction methods known in the art.

It has been found that the size and the size distribution of the nano- and/or microparticles can be conveniently controlled in this process, e.g. by varying the energy of agitation during the addition of the aqueous phase, or by varying the composition and/or the viscosity of the aqueous surfactant phase.

After the suspension of nano- and/or microparticles has been formed, the nano- and/or microparticles can be isolated from the liquid phase and dried via conventional methods, including e.g. extraction, spray drying, fluid bed drying, freeze drying, centrifugation, evaporation and/or filtration. These methods can also be used to remove residues of the solvents S1 and/or S2, if necessary. Volatile solvents can be conveniently removed from the particles via evaporation. Less volatile solvents can be removed by other methods established in the art, such as extraction. Washing steps can also be added to the process of the invention as needed. A particularly convenient step in order to obtain a dry, reconstitutable powder containing the nano- and/or microparticles is lyophilisation.

The nano- and/or microparticles containing the active principle can be conveniently stored e.g. as dry powders.

In a further aspect, the present invention provides the nano- and/or microparticles as defined above, including their preferred embodiments, for use as a medicament, and specifically for use as a medicament for treatment of the human or animal body by therapy. In particular, the invention provides the nano- and/or microparticles as defined above, including their preferred embodiments, for use in the treatment or prevention of a mental disorder, preferably in the treatment of a neuropsychiatric disorder, and more preferably in the treatment or prevention of schizophrenia or bipolar disorder, or of symptoms associated with schizophrenia or bipolar disorder. Preferably, the medicament is a sustained release medicament, or a depot medicament. Such a depot medicament is a medicament which contains an amount of the active principle that is sufficient to provide a therapeutic plasma level of the active principle over an extended period of time, such as 1 week or more, preferably 2 weeks or more in the body of the subject to which the depot medicament is administered. To that extent, the invention also provides the nano- and/or microparticles as defined above, including their preferred embodiments, for use in the treatment or prevention of a mental disorder, preferably in the treatment of a neuropsychiatric disorder, and more preferably in the treatment or prevention of schizophrenia or bipolar disorder, or of symptoms associated with schizophrenia or bipolar disorder, wherein the nano- and/or microparticles are to be administered in intervals of at least 1 week, preferably at least 2 weeks, between consecutive administrations. Typically, the treatment involving the administration in these intervals extends over periods of several months or years, i.e. more than one month, or more than one year. The nano- and/or microparticles can be administered by any convenient route, and are advantageously administered via the parenteral route, preferably via parenteral injection, and in particular via subcutaneous or intramuscular injection.

A related aspect concerns the use of the nano- and/or microparticles as defined above, including their preferred embodiments, for the preparation of a medicament. The medicament can be used in the treatment or prevention of a mental disorder, preferably in the treatment of a neuropsychiatric disorder, and more preferably in the treatment or prevention of schizophrenia or bipolar disorder, or of symptoms associated with schizophrenia or bipolar disorder. Preferably, the medicament is a sustained release medicament, or a depot medicament. Such a depot medicament is a medicament which contains an amount of the active principle that is sufficient to provide a therapeutic plasma level of the active principle over an extended period of time, such as 1 week or more, preferably 2 weeks or more in the body of the subject to which the depot medicament is administered. To that extent, the invention also provides the nano- and/or microparticles as defined above, including their preferred embodiments, for use in the manufacture of a medicament for the treatment or prevention of a mental disorder, preferably in the treatment of a neuropsychiatric disorder, and more preferably in the treatment or prevention of schizophrenia or bipolar disorder, or of symptoms associated with schizophrenia or bipolar disorder, wherein the medicament is to be administered in intervals of at least 1 week, preferably at least 2 weeks, between consecutive administrations. Typically, the treatment involving the administration in these intervals extends over periods of several months or years, i.e. more than one month, or more than one year. The medicament can be administered by any convenient route, and is advantageously administered via the parenteral route, preferably via parenteral injection, and in particular via subcutaneous or intramuscular injection.

A further aspect of the invention concerns a pharmaceutical formulation comprising the nano- and/or microparticles as defined above, including their preferred embodiments, optionally in combination with an additional pharmaceutically acceptable excipient. The formulation can be used in the treatment or prevention of a mental disorder, preferably in the treatment of a neuropsychiatric disorder, and more preferably in the treatment or prevention of schizophrenia or bipolar disorder, or of symptoms associated with schizophrenia or bipolar disorder. Preferably, the pharmaceutical formulation is a sustained release formulation or a depot formulation. Such a depot formulation is a formulation which contains an amount of the active principle that is sufficient to provide a therapeutic plasma level of the active principle over an extended period of time, such as 1 week or more, preferably 2 weeks or more in the body of the subject to which the depot formulation is administered. To that extent, the invention also provides the pharmaceutical formulation as defined above, for use in the treatment or prevention of a mental disorder, preferably in the treatment of a neuropsychiatric disorder, and more preferably in the treatment or prevention of schizophrenia or bipolar disorder, or of symptoms associated with schizophrenia or bipolar disorder, wherein the formulation is to be administered in intervals of at least 1 week, preferably at least 2 weeks, between consecutive administrations. Typically, the treatment involving the administration in these intervals extends over periods of several months or years, i.e. more than one month, or more than one year. The formulation can be administered by any convenient route, and is advantageously administered via the parenteral route, preferably via parenteral injection, and in particular via subcutaneous or intramuscular injection.

Thus, in accordance with a particularly preferred embodiment, the present invention provides a pharmaceutical formulation comprising nano- and/or microparticles, which nano- and/or microparticles comprise asenapine as an active agent dispersed in a polymer matrix, wherein the polymer matrix comprises a poly(lactide-co-glycolide) copolymer, and wherein the asenapin is dispersed in the polymer matrix in an amount of at least 10 wt. %, calculated as the free base, in particular at least 15 wt. %, based on the total weight of the nano- and/or microparticles.

In addition to the nano- and/or microparticles, the pharmaceutical formulation in accordance with the present invention may contain one or more pharmaceutically acceptable excipients. Exemplary pharmaceutically acceptable excipients that may be used in the pharmaceutical formulation of the invention are selected from carriers, vehicles, diluents, in particular water, e.g. in the form of water for injection, or in the form of a physiological salt solution, other solvents such as monohydric alcohols, such as ethanol, or isopropanol, and polyhydric alcohols such as glycols and edible oils such as soybean oil, coconut oil, olive oil, safflower oil, cottonseed oil, oily esters such as ethyl oleate, isopropyl myristate; binders, adjuvants, solubilizers, thickening agents, stabilizers, disintegrants, lubricating agents, buffering agents, emulsifiers, wetting agents, suspending agents, sweetening agents, colourants, flavours, preservatives, antioxidants, processing agents, drug delivery modifiers and enhancers such as calcium phosphate, magnesium stearate, talc, monosaccharides, disaccharides, starch, gelatine, cellulose, methylcellulose, sodium carboxymethyl cellulose, dextrose, hydroxypropyl-β-cyclodextrin, polyvinylpyrrolidone or polyethylene glycol. Other suitable pharmaceutically acceptable excipients are described in Remington's Pharmaceutical Sciences, 15th Ed., Mack Publishing Co., New Jersey (1991). It will be understood that the excipient(s) need to be selected in accordance with the planned route of administration. A preferred formulation in accordance with the present invention is a liquid formulation suitable for parenteral injection, comprising the nano- and/or microparticles in accordance with the invention in dispersed form. Suitable liquid phases for liquid formulations for parenteral administration are well known in the art. The liquid phase typically comprises water, in particular in the form of water for injection or in the form of a a physiological salt solution, and optionally further adjuvants selected e.g. from a buffer, an acid or a base for adjusting the pH, a dispersing agent, a surfactant, an agent for adjusting the viscosity, and combinations thereof. Exemplary components of such a liquid formulation are water for injection, and Tween 20 or Tween 80 as surfactants, sodium carboxymethyl cellulose, mannitol, dextran, acids or bases like acetic acid, citric acid, or NaOH, or salts like NaCl.

The pharmaceutical formulation, in particular the formulation for parenteral injection in accordance with the invention preferably contains the nano- and/or microparticles in an amount of 5 to 60 wt %, based on the total weight of the formulation, more preferably of 10 to 50 wt %.

In a related embodiment, the invention also provides a kit containing, in separate containers or compartments, (i) the nano- and/or microparticles in accordance with the invention e.g. in the form of a dry powder and (ii) water for injection or a physiological solution, preferably a salt solution for reconstitution of the powder.

In line with the above, the nano- and/or microparticles of the present invention, including their preferred embodiments, can be suitably used in a method for the treatment or prevention of a mental disorder, preferably in the treatment of a neuropsychiatric disorder, and more preferably in the treatment or prevention of schizophrenia or bipolar disorder, or of symptoms associated with schizophrenia or bipolar disorder, said method comprising administering the nano- and/or microparticles of the present invention, or a pharmaceutical formulation comprising them, to a subject in need thereof. It will be understood that the nano- and/or microparticles should be administered in the context of this method, either per se or as part of a pharmaceutical formulation, in a therapeutically effective amount. Preferably, the method involves the administration of the nano- and/or microparticles of the present invention, or a pharmaceutical formulation comprising them, in intervals of at least 1 week, preferably at least 2 weeks, between consecutive administrations. Typically, the method involves the administration in these intervals extends over periods of several months or years, i.e. more than one month, or more than one year. Advantageously, the method involves the administration of the nano- and/or microparticles of the present invention, or a pharmaceutical formulation comprising them, via the parenteral route, preferably via parenteral injection, and in particular via subcutaneous or intramuscular injection.

In a further aspect, the present invention provides the nano- and/or microparticles as defined above, including their preferred embodiments, which nano- and/or microparticles are adapted for parenteral administration to a subject, in particular for parenteral injection. Typically, the administration or injection occurs intramuscularly or subcutaneously.

As regards the administration of the nano- and/or microparticles in accordance with the invention (including the nano- and/or microparticles for use in treatment and prevention as mentioned above, and also the nano- and/or microparticles contained in a medicament or a pharmaceutical formulation as referred to above), it had been noted above that the administration occurs advantageously via the parenteral route. Preferred is the intramuscular or subcutaneous administration, and particular preferred to intramuscular injection. For example, the injection may be made in the gluteal or deltoid muscles.

The dose of the nano- and/or microparticles, the medicament or the pharmaceutical preparation to be administered will be determined by the attending physician and clinical factors. As is well known in the medical arts, dosages for any one patient depends upon many factors, including factor such as the patient's size, body surface weight, age, sex, general health, individual response of the patient to be treated, and the severity of the disorder to be treated. For example, the dose may be selected such that the administered amount of active principle, calculated as the asenapine free base, ranges from 30 to 1000 mg.

As will be understood, the mention of a treatment or prevention herein generally refers to the treatment or prevention of a disorder in an animal, preferably a mammal, and in particular a human subject. Similarly, any reference to the administration of the nano- and/or microparticles of the invention, or of a medicament or a pharmaceutical formulation comprising them generally refers to the administration to an animal, preferably a mammal, and most preferably to a human subject.

Moreover, the term “treatment” of a disorder or disease as used herein is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease.

The term “prevention” of a disorder or disease as used herein is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of compounds of the present invention before any clinical and/or pathological symptoms are diagnosed or determined or can be diagnosed or determined by the attending physician.

Important aspects of the invention disclosed above shall in addition be summarized below:

1. Nano- and/or microparticles comprising asenapine or a pharmaceutically acceptable salt thereof, wherein said asenapine or said pharmaceutically acceptable salt thereof is embedded in a polymer matrix or encapsulated by a polymer shell, wherein the polymer matrix or the polymer shell comprises a polymer selected from polylactide, polyglycolide, and polyester copolymers comprising copolymerized units of lactic acid and/or glycolic acid.
2. The nano- and/or microparticles according to item 1, wherein the content of asenapine or the pharmaceutically acceptable salt thereof is at least 10 wt. %, based on the total weight of the particles.
3. The nano- and/or microparticles according to item 1, wherein the content of asenapine or the pharmaceutically acceptable salt thereof is at least 15 wt. %, based on the total weight of the particles.
4. The nano- and/or microparticles according to item 1, wherein the content of asenapine or the pharmaceutically acceptable salt thereof is at least 20 wt. %, based on the total weight of the particles.
5. The nano- and/or microparticles according to item 1, wherein the content of asenapine or the pharmaceutically acceptable salt thereof is not more than 50% wt. %, based on the total weight of the particles.
6. The nano- and/or microparticles of any of items 1 to 5, which comprise the asenapine in the form of the asenapine free base, or in the form of a salt selected from hydrochloride, hydrobromide, hydroiodide, sulfate, nitrate, phosphate, carbonate, hydrogencarbonate, perchlorate, acetate, propionate, butyrate, pentanoate, hexanoate, heptanoate, octanoate, cyclopentanepropionate, undecanoate, lactate, maleate, oxalate, fumarate, tartrate, malate, citrate, nicotinate, benzoate, salicylate, pamoate, ascorbate, methanesulfonate, ethanesulfonate, 2-hydroxyethanesulfonate, benzenesulfonate, p-toluenesulfonate (tosylate), 2-naphthalenesulfonate, 3-phenylsulfonate, camphorsulfonate, aspartate and glutamate salt.
7. The nano- and/or microparticles of any of items 1 to 5, which comprise the asenapine in the form of a maleate salt.
8. The nano- and/or microparticles of any of items 1 to 7, wherein the asenapine or the pharmaceutically acceptable salt thereof is dispersed as an amorphous or crystalline solid in a polymer matrix.
9. The nano- and/or microparticles of any of items 1 to 8, wherein the polymer matrix or polymer shell comprises a polymer selected from the group consisting of a polyglycolide homopolymer, a polylactide homopolymer, a copolymer of glycolide and lactide, a copolymer of glycolide and tetramethylglycolide, a copolymer of glycolide and δ-valerolactone, a copolymer of glycolide and ε-caprolactone, a copolymer of glycolide and trimethylene carbonate, a copolymer of lactide and tetramethylglycolide, a copolymer of lactide and δ-valerolactone, a copolymer of lactide and ε-caprolactone, a copolymer of lactide and trimethylene carbonate, a copolymer of glycolide and ethylene glycol, and a copolymer of lactide and ethylene gylcol.
10. The nano- and/or microparticles of item 9, wherein the polymer matrix or polymer shell comprises a poly(lactide-co-glycolide) copolymer.
11. The nano- and/or microparticles of any of items 1 to 10, which have a particle size, determined by laser scattering, within the size range of 50 nm to 300 μm.
12. The nano- and/or microparticles of any of items 1 to 10, which have a volume weighted mean diameter of 1 μm to 125 μm.
13. The nano- and/or microparticles of any of items 1 to 12, which are obtainable by a process comprising the steps of
a) providing a solution of a polymer in an organic solvent S1 having limited water solubility; wherein said polymer is selected from the group consisting of polylactide, polyglycolide, and polyester copolymers comprising copolymerized units of lactic acid and/or glycolic acid;
b) combining the solution provided in step a) with asenapine or a pharmaceutically acceptable salt thereof by
   b1) dispersing the asenapine or the pharmaceutically acceptable salt thereof in the solution provided in step a), or
   b2) providing a solution or dispersion of the asenapine or the pharmaceutically acceptable salt thereof in an organic solvent S2 and combining the solution or dispersion with the solution provided in step a),
to provide an organic phase which comprises dissolved polymer and asenapine or a pharmaceutically acceptable salt thereof dissolved or dispersed therein;
c) agitating the organic phase provided in step b) in a vessel and adding an aqueous surfactant solution to the agitated organic phase in a volume ratio of at least 2:1 in terms of the total volume of the aqueous surfactant solution to the total volume of the organic phase provided in step b), thus causing the formation of a dispersion containing a continuous aqueous phase and a discontinuous organic phase; and
d) allowing the formation, typically spontaneous formation, of a suspension of the nano- and/or microparticles via transfer of organic solvent from the discontinuous organic phase into the aqueous surfactant phase directly after the dispersion has been formed.
   14. A process for the production of the nano- and/or microparticles of any of items 1 to 12, said process comprising the steps of
a) providing a solution of a polymer in an organic solvent S1 having limited water solubility; wherein said polymer is selected from the group consisting of polylactide, polyglycolide, and polyester copolymers comprising copolymerized units of lactic acid and/or glycolic acid;
b) combining the solution provided in step a) with asenapine or a pharmaceutically acceptable salt thereof by
   b1) dispersing the asenapine or the pharmaceutically acceptable salt thereof in the solution provided in step a), or
   b2) providing a solution or dispersion of the asenapine or the pharmaceutically acceptable salt thereof in an organic solvent S2 and combining the solution or dispersion with the solution provided in step a),
to provide an organic phase which comprises dissolved polymer and asenapine or a pharmaceutically acceptable salt thereof dissolved or dispersed therein;
c) agitating the organic phase provided in step b) in a vessel and adding an aqueous surfactant solution to the agitated organic phase in a volume ratio of at least 2:1 in terms of the total volume of the aqueous surfactant solution to the total volume of the organic phase provided in step b), thus causing the formation of a dispersion containing a continuous aqueous phase and a discontinuous organic phase; and
d) allowing the formation, typically spontaneous formation, of a suspension of the nano- and/or microparticles via transfer of organic solvent from the discontinuous organic phase into the aqueous surfactant phase directly after the dispersion has been formed.
   15. The process of item 14, wherein the volume ratio of the total volume of the aqueous surfactant solution added in step c) to the total volume of the organic phase provided in step b) prior to the addition is in the range of 2:1 to 5:1.
   16. The process of item 14 or 15, wherein the solvent S1 is selected from alkyl acetates, alkyl formates, and mixtures of two or more thereof.
   17. The process of any of items 14 to 16, wherein the solution provided in step a) is combined with asenapine or a pharmaceutically acceptable salt thereof by b2) providing a solution or dispersion of the asenapine or the pharmaceutically acceptable salt thereof in an organic solvent S2 and combining the solution or dispersion with the solution provided in step a).
   18. The process of item 17, wherein the organic solvent S2 is selected from alkyl esters of benzoic acid, in particular the methyl or ethyl ester, aryl esters of benzoic acid, benzyl alcohol, dimethyl sulfoxide (DMSO), N-methyl pyrrolidone (NMP), glycofurol and mixtures thereof.
   19. The process of any of items 14 to 18, wherein the aqueous surfactant phase is added to the organic phase in step b) by adding it to the total volume of the organic phase under stirring such that the content of the surfactant solution in the combined surfactant solution and organic phase gradually increases until the addition is completed, and the addition takes place over a period of time of 5 s to 5 min, preferably 10 s to 2 min.
   20. The nano- and/or microparticles of any of items 1 to 13, which are adapted for parenteral administration to a subject.
   21. The nano- and/or microparticles of item 20, which are adapted for subcutaneous or intramuscular injection.
   22. A pharmaceutical formulation comprising the nano- and/or microparticles of any of items 1 to 13, optionally in combination with a pharmaceutically acceptable excipient.
   23. The pharmaceutical formulation of item 22 for use in the treatment or prevention of a mental disorder.
   24. The pharmaceutical formulation of item 23, wherein the mental disorder is selected from schizophrenia, bipolar disorder, and symptoms associated with schizophrenia or bipolar disorder.
   25. The pharmaceutical formulation of any of items 22 to 24, which is to be administered by the parenteral route.
   26. The pharmaceutical formulation of item 25, which is to be administered via subcutaneous or intramuscular injection.
   27. The pharmaceutical formulation of any of items 22 to 26, which is to be administered in intervals of at least 1 week between consecutive administrations.
   28. The pharmaceutical formulation of any of items 22 to 26, which is to be administered in intervals of at least 2 weeks between consecutive administrations.
   29. The nano- and/or microparticles of any of items 1 to 13 for use as a medicament.
   30. The nano- and/or microparticles according to any of items 1 to 13 for use in the treatment or prevention of a mental disorder.
   31. The nano- and/or microparticles of item 30, wherein the mental disorder is selected from schizophrenia, bipolar disorder, and symptoms associated with schizophrenia or bipolar disorder.
   32. The nano- and/or microparticles of any of items 29 to 31, which are to be administered by the parenteral route.
   33. The nano- and/or microparticles of item 32, which are to be administered via subcutaneous or intramuscular injection.
   34. The nano- and/or microparticles of any of items 29 to 33, which are to be administered in intervals of at least 1 week between consecutive administrations.
   35. The nano- and/or microparticles of any of items 29 to 33, which are to be administered in intervals of at least 2 weeks between consecutive administrations.
   36. Use of the nano- and/or microparticles of any of items 1 to 13 for the preparation of a medicament for the treatment or prevention of a mental disorder.
   37. The use of item 36, wherein the mental disorder is selected from schizophrenia, bipolar disorder, and symptoms associated with schizophrenia or bipolar disorder.
   38. The use of items 36 or 37, wherein the medicament is to be administered by the parenteral route.
   39. The use of item 38, wherein the medicament is to be administered via subcutaneous or intramuscular injection.
   40. The use of any of items 36 to 39, wherein the medicament is to be administered in intervals of at least 1 week between consecutive administrations.
   41. The use of any of items 36 to 39, wherein the medicament is to be administered in intervals of at least 2 weeks between consecutive administrations.
   42. A method of treating or preventing a mental disorder, comprising administering the nano- and/or microparticles of any of items 1 to 13 or the pharmaceutical formulation of item 22 to a subject in need thereof.
   43. The method of item 42, wherein the mental disorder is selected from schizophrenia, bipolar disorder, and symptoms associated with schizophrenia or bipolar disorder.
   44. The method of item 42 or 43, wherein the nano- and/or microparticles or the pharmaceutical formulation are/is administered by the parenteral route.
   45. The method of item 44, wherein the nano- and/or microparticles or the pharmaceutical formulation are/is administered via subcutaneous or intramuscular injection.
   46. The method of any of items 42 to 45, wherein the nano- and/or microparticles or the pharmaceutical formulation are/is administered in intervals of at least 1 week between consecutive administrations.
   47. The method of any of items 42 to 45, wherein the nano- and/or microparticles or the pharmaceutical formulation are/is administered in intervals of at least 2 weeks between consecutive administrations.
   48. A kit containing, in separate containers or compartments, (i) the nano- and/or microparticles according to any of claims 1 to 13 in the form of a dry powder and (ii) water for injection or a physiological solution for reconstitution of the powder.

### EXAMPLES

### Determination of the Active Principle Content in Nano- and/or Microparticles

The active principle content (“content”) measured in a sample of the nano- and/or microparticles is the mass of the asenapine free base/mass sample·100%. The content was determined by dissolving drug loaded nano and/or microparticles in acetonitrile, and determining the concentration of the active principle via reverse phase high-performance liquid chromatography (RP-HPLC).

Determination of the Theoretical Active Principle Content in Nano- and/or Microparticles

The theoretical active principle content (“theoretical content”) reflects the maximum active principle content. It is calculated from the masses of all educts present in the final formulation. Thus the theoretical content is defined as mass of the asenapine base divided by the sum of mass asenapine formulation+mass polymer+mass excipients·100%. The mass of excipients includes surfactants, as well as the residual organic solvent(s) used for the solubilization of asenapine as well as the polymers. The total mass of excipients present in the nano and/or microparticles is approximated to about 5% of the sum of the masses of asenapine formulation and polymer. Thus, the mass of excipients is defined as 0.05·(m asenapine formulation+m polymer)

Determination of the Encapsulation Efficiency

The encapsulation efficiency for the active principle embedded in the polymer matrix and the active principle encapsulated by a polymer shell is defined as quotient of the content and theoretical content·100%

### Mean Diameter

The mean diameter is calculated as volume weighted mean diameter that represents the arithmetic mean size in volume % mode (D(4,3)).

### Example 1

1.5 g of Polymer Resomer® RG755S and 1.5 g of Polymer Resomer® RG753S were dissolved in 10 ml ethyl acetate and transferred to a double-walled glass vessel (inside height 16.0 cm, inside diameter 3.4 cm). The API solution containing 1000.0 mg asenapine maleate in 3.5 ml benzyl alcohol was dispersed in the polymer solution by means of a mechanical agitator (Dispermat FT, VMA-Getzmann GmbH, Germany, equipped with a 2.5 cm dissolver disc) for 20 min at 3000 rpm and for 25 min at 4000 rpm and for 0.5 min at 3000 rpm at room temperature.

80 mL PVA solution (2.0% (w/v)) in 50 mmol phosphate buffer pH 8.0 was added as continuous phase during agitation at 3000 rpm.

After about 60 seconds of agitation, the suspension of microparticles was transferred to a 3 L beaker and 500 mL PVA solution (1% (w/v)) in 50 mmol phosphate buffer pH 8.0 was added. The suspension was stirred magnetically. After 30 min another 500 mL PVA solution (1% (w/v)) in 50 mmol phosphate buffer pH 8.0 containing 20% (v/v) ethanol was added to the suspension.

The organic solvents ethyl acetate and benzyl alcohol were removed by extraction.

After 30 min microparticles were collected by filtration. Subsequently, microparticles were diluted by addition of 500 mL poloxamer 188 solution (10% (w/v)) in 50 mmol phosphate pH 8.0. Another 300 mL of that poloxamer 188 solution was added after 30 min. A mixture of 100 mL ethanol and 100 mL poloxamer 188 solution was added after 30 min and after 60 min, and finally microparticles were separated by filtration and concentrated to the desired volume. The suspension was stored frozen until lyophilisation.

The lyophilisate, resuspended in water contained microparticles with active principle content of 14.4% and with an encapsulations efficiency of 77.5%. The microparticles had a mean diameter of 24.4 μm.

The in-vitro release profile of the formulation was measured. 50 mg microparticles were weighed into a 1000 mL dissolution vessel and 500 mL release medium was added. The release medium consisted of 50 mM HEPES buffer pH 7.0 containing 100 mM NaCl, 0.05% (w/v) Tween 80 and 0.01% (w/v) sodium azid. The release of each example was determined as duplicate by means of a Dissolution tester (Sotax) at 37° C. and at a stirring rate of 100 rpm. Samples were withdrawn by menas of syringe equipped with a pre-filter at certain timepoints. The content of asenapine in samples of the release medium was determined by liquid chromatography. Reverse phase high-performance liquid chromatography (RP-HPLC) was carried out on an Alliance 2695 Separations Module (Waters, Eschborn, Germany) equipped with a Gemini C18 column (4.6×150 mm, 3 μm, Phenomenex, Aschaffenburg, Germany). LC was conducted in isocratic mode with a mobile phase consisting of 40% methanol, 30% acetonitrile, 30% ultrapure water, 0.056% (w/v) potassium hydroxide. The pH was adjusted by addition of diluted phosphoric acid to pH 7.6 at 20° C. Chromatographic separation was carried out at a flow rate of 0.9 mL/min with a run time of 15 minutes at 40° C. Elution profile was monitored with a variable UV detector at 228 nm under the control and evaluation of Empower 2 software (Waters, Eschborn, Germany).

FIG. 1 shows the in-vitro release profile of Example 1.

### Example 2

1.5 g of Polymer Resomer® RG755S and 1.5 g of Polymer Resomer® RG753H were dissolved in 10 ml ethyl acetate and transferred to a double-walled glass vessel (inside height 16.0 cm, inside diameter 3.4 cm). The API solution containing 1.0 g asenapine maleate in 3.5 ml benzyl alcohol was dispersed in the polymer solution by means of a mechanical agitator (Dispermat FT, VMA-Getzmann GmbH, Germany, equipped with a 2.5 cm dissolver disc) for 20 min at 3000 rpm and for 25 min at 4000 rpm at room temperature.

80 mL PVA solution (1.0% (w/v)) in 50 mmol phosphate buffer pH 8.0 was added as continuous phase during agitation at 3000 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 1.

The lyophilisate, resuspended in water contained microparticles with active principle content of 16.0% and with an encapsulations efficiency of 85.8%. The microparticles had a mean diameter of 48.7 μm.

The in-vitro release profile was measured as described for Example 1 (FIG. 2).

### Example 3

1.5 g asenapine maleate was transferred to a double-walled glass vessel (inside height 16.0 cm, inside diameter 3.4 cm). 1.5 g of Polymer Resomer® RG755S and 1.5 g of Polymer Resomer® RG753H were dissolved in 10 ml ethyl formate and transferred to the double-walled glass vessel. The solid API asenapine maleate was dispersed in the polymer solution by means of a mechanical agitator (Dispermat FT, VMA-Getzmann GmbH, Germany, equipped with a 2.5 cm dissolver disc) for 14 min at 3000 rpm at room temperature.

80 mL PVA solution (1.0% (w/v)) in 50 mmol phosphate buffer pH 8.0 was added as continuous phase during agitation at 3000 rpm.

After about 60 seconds of agitation, the suspension of microparticles was transferred to a 3 L beaker and 1 L PVA solution (1% (w/v)) in 50 mmol phosphate buffer pH 8.0 was added. The suspension was stirred magnetically. The organic solvent ethyl formate was removed by extraction. After 60 min 100 mL ethanol was added.

After 1 hour microparticles were collected by filtration. Microparticles were diluted by addition of 1 L poloxamer 188 solution (4% (w/v)) in 50 mmol phosphate pH 8.0. After 30 min the microparticles were separated by filtration and concentrated to the desired volume. The suspension was stored frozen until lyophilisation.

The lyophilisate, resuspended in water contained microparticles with active principle content of 19.5% and with an encapsulations efficiency of 78.9%. The microparticles had a mean diameter of 48.2 μm.

The in-vitro release profile was measured as described for Example 1 (FIG. 3).

### Example 4

1.5 g of Polymer Resomer® RG755S and 1.5 g of Polymer Resomer® RG753H were dissolved in 10 ml ethyl acetate and transferred to a double-walled glass vessel (inside height 16.0 cm, inside diameter 3.4 cm). A solution containing 1000 mg asenapine maleate in 3.2 mL benzyl alcohol was dispersed in the polymer solution by means of a mechanical agitator (Dispermat FT, VMA-Getzmann GmbH, Germany, equipped with a 2.5 cm dissolver disc) for 20 min at 3000 rpm and for 25 min at 4000 rpm at room temperature.

80 mL PVA solution (1.0% (w/v)) in 50 mmol phosphate buffer pH 8.0 was added as continuous phase during agitation at 3000 rpm.

After about 60 seconds of agitation, the suspension of microparticles was transferred to a 3 L beaker and 1 L PVA solution (1% (w/v)) in 50 mmol phosphate buffer pH 8.0 was added. The suspension was stirred magnetically. After 60 min another volume of 300 mL PVA solution was added. After 30 min a mixture of 100 mL PVA solution (1% (w/v)) in 50 mmol phosphate buffer pH 8.0 and 100 mL ethanol was transferred to the beaker. This was repeated twice. Subsequently, 100 mL ethanol was added after 30 min and another 100 mL after 60 min. The organic solvents ethyl acetate and benzyl alcohol were removed by extraction.

After 4 hours microparticles were collected by filtration. Subsequently, microparticles were diluted by addition of 1 L poloxamer 188 solution (4% (w/v)) in 50 mmol phosphate pH 8.0. Finally the microparticles were separated by filtration and concentrated to the desired volume. The suspension was stored frozen until lyophilisation.

The lyophilisate, resuspended in water contained microparticles with active principle content of 16.0% and with an encapsulations efficiency of 88.7%. The microparticles had a mean diameter of 83.1 μm.

The particle size distribution of Example 4 is shown in FIG. 4.

### Example 5

1.5 g of Polymer Resomer® RG755S and 1.5 g of Polymer Resomer® RG753H were dissolved in 10 ml ethyl acetate and transferred to a double-walled glass vessel (inside height 16.0 cm, inside diameter 3.4 cm). The API solution containing 1000 mg asenapine maleate in 3.5 mL benzyl alcohol was dispersed in the polymer solution by means of a mechanical agitator (Dispermat FT, VMA-Getzmann GmbH, Germany, equipped with a 2.5 cm dissolver disc) for 20 min at 3000 rpm and for 25 min at 4000 rpm at room temperature.

80 mL PVA solution (2.0% (w/v)) in 50 mmol phosphate buffer pH 8.0 was added as continuous phase during agitation at 3000 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 1.

The lyophilisate, resuspended in water contained microparticles with active principle content of 15.7% and with an encapsulations efficiency of 84.4%. The microparticles had a mean diameter of 42.8 μm.

The in-vitro release profile was measured as described for Example 1 (FIG. 5).

### Example 6

2.0 g asenapine maleate was transferred to a double-walled glass vessel (inside height 16.0 cm, inside diameter 3.4 cm). 1.5 g of Polymer Resomer® RG755S and 1.5 g of Polymer Resomer® RG753H were dissolved in 12 ml ethyl formate and transferred to the double-walled glass vessel. The solid API asenapine maleate was dispersed in the polymer solution by means of a mechanical agitator (Dispermat FT, VMA-Getzmann GmbH, Germany, equipped with a 2.5 cm dissolver disc) for 10 min at 2500 rpm and for 8 min at 3000 rpm at room temperature.

80 mL PVA solution (1.0% (w/v)) in 50 mmol phosphate buffer pH 8.0 was added as continuous phase during agitation at 3000 rpm.

After about 60 seconds of agitation, the suspension of microparticles was transferred to a 3 L beaker and 1 L PVA solution (1% (w/v)) in 50 mmol phosphate buffer pH 8.0 was added. The suspension was stirred magnetically. The organic solvent ethyl formate was removed by extraction. After 60 min 100 mL ethanol was added.

After 1 hour microparticles were collected by filtration. Microparticles were diluted by addition of 1 L poloxamer 188 solution (4% (w/v)) in 50 mmol phosphate pH 8.0. After 30 min the micro particles were separated by filtration and concentrated to the desired volume. The suspension was stored frozen until lyophilisation.

The lyophilisate, resuspended in water contained microparticles with active principle content of 21.4% and with an encapsulations efficiency of 72.6%. The microparticles had a mean diameter of 58.7 μm.

The in-vitro release profile was measured as described for Example 1 (FIG. 6).

### Example 7

2.0 g asenapine maleate was transferred to a double-walled glass vessel (inside height 16.0 cm, inside diameter 3.4 cm). 1.5 g of Polymer Resomer® RG755S and 1.5 g of Polymer Resomer® RG753H were dissolved in 12 ml ethyl acetate and transferred to the double-walled glass vessel. The solid API asenapine maleate was dispersed in the polymer solution by means of a mechanical agitator (Dispermat FT, VMA-Getzmann GmbH, Germany, equipped with a 2.5 cm dissolver disc) for 13 min at 2500 rpm and for 15 min at 3000 rpm at room temperature.

80 mL PVA solution (1.0% (w/v)) in 50 mmol phosphate buffer pH 8.0 was added as continuous phase during agitation at 3000 rpm.

After about 60 seconds of agitation, the suspension of microparticles was transferred to a 3 L beaker and 1 L PVA solution (1% (w/v)) in 50 mmol phosphate buffer pH 8.0 was added. After 60 min 100 mL ethanol and after 90 min 50 mL ethanol was added. After 2 hours micro-particles were collected by filtration. Extraction of ethyl acetate was performed as described for Example 6.

The lyophilisate, resuspended in water contained micro particles with active principle content of 21.9% and with an encapsulations efficiency of 75.1%. The micro-particles had a mean diameter of 106.7 μm.

The in-vitro release profile was measured as described for Example 1 (FIG. 7).

### Example 8

1.5 g of Polymer Resomer® RG755S and 1.5 g of Polymer Resomer® RG753H were dissolved in 10 ml ethyl acetate and transferred to a double-walled glass vessel (inside height 16.0 cm, inside diameter 3.4 cm). The API solution containing 1.0 g asenapine maleate in 3.2 ml benzyl alcohol was dispersed in the polymer solution by means of a mechanical agitator (Dispermat FT, VMA-Getzmann GmbH, Germany, equipped with a 2.5 cm dissolver disc) for 20 min at 3000 rpm and for 25 min at 4000 rpm and for 0.5 min at 3000 rpm at room temperature.

80 mL PVA solution (1.0% (w/v)) in 50 mmol phosphate buffer pH 8.0 was added as continuous phase during agitation at 3000 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 1.

The lyophilisate, resuspended in water contained microparticles with active principle content of 15.2% and with an encapsulations efficiency of 84.2%. The microparticles had a mean diameter of 54.4 μm.

### Example 9

1.5 g of Polymer Resomer® RG755S and 1.5 g of Polymer Resomer® RG753H were dissolved in 10 ml ethyl acetate and transferred to a double-walled glass vessel (inside height 16.0 cm, inside diameter 3.4 cm). The API solution containing 2.0 g asenapine maleate in 6.0 ml benzyl alcohol was dispersed in the polymer solution by means of a mechanical agitator (Dispermat FT, VMA-Getzmann GmbH, Germany, equipped with a 2.5 cm dissolver disc) for 24 min at 2500 rpm and for 11 min at 3000 rpm and for 45 min at 4000 rpm at room temperature.

80 mL PVA solution (1.0% (w/v)) in 50 mmol phosphate buffer pH 8.0 was added as continuous phase during agitation at 3000 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 1.

The lyophilisate, resuspended in water contained micro particles with active principle content of 25.2% and with an encapsulations efficiency of 87.1%. The microparticles had a mean diameter of 40.4 μm.

### Example 10

1.5 g of Polymer Resomer® RG755S and 1.5 g of Polymer Resomer® RG753H were dissolved in 7 ml ethyl acetate and transferred to a double-walled glass vessel (inside height 16.0 cm, inside diameter 3.4 cm). The API solution containing 2.0 g asenapine maleate in 6.0 ml benzyl alcohol was dispersed in the polymer solution by means of a mechanical agitator (Dispermat FT, VMA-Getzmann GmbH, Germany, equipped with a 2.5 cm dissolver disc) for 10 min at 2500 rpm and for 10 min at 3000 rpm and for 25 min at 4000 rpm at room temperature.

80 mL PVA solution (1.0% (w/v)) in 50 mmol phosphate buffer pH 8.0 was added as continuous phase during agitation at 3000 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 1.

The lyophilisate, resuspended in water contained microparticles with active principle content of 26.5% and with an encapsulations efficiency of 90.8%. The microparticles had a mean diameter of 64.8 μm.

### Example 11

1.5 g of Polymer Resomer® RG755S and 1.5 g of Polymer Resomer® RG753H were dissolved in 10 ml ethyl acetate and transferred to a double-walled glass vessel (inside height 16.0 cm, inside diameter 4.6 cm). The API solution containing 2.0 g asenapine maleate in 4.0 ml benzyl alcohol was dispersed in the polymer solution by means of a mechanical agitator (Dispermat FT, VMA-Getzmann GmbH, Germany, equipped with a 3.0 cm dissolver disc) for 15 min at 2500 rpm and for 25 min at 3500 rpm and for 1 min at 2500 rpm at room temperature.

80 mL PVA solution (1.0% (w/v)) in 50 mmol phosphate buffer pH 8.0 was added as continuous phase during agitation at 2500 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 1.

The lyophilisate, resuspended in water contained microparticles with active principle content of 28.7% and with an encapsulations efficiency of 99.7%. The microparticles had a mean diameter of 78.2 μm.

### Example 12

1.5 g of Polymer Resomer® RG755S and 1.5 g of Polymer Resomer® RG753H were dissolved in 10 ml ethyl acetate and transferred to a double-walled glass vessel (inside height 16.0 cm, inside diameter 4.6 cm). The API solution containing 2.0 g asenapine maleate in 4.0 ml benzyl alcohol was dispersed in the polymer solution by means of a mechanical agitator (Dispermat FT, VMA-Getzmann GmbH, Germany, equipped with a 3.0 cm dissolver disc) for 15 min at 2500 rpm and for 5 min at 3500 rpm and for 0.5 min at 2500 rpm at room temperature.

80 mL PVA solution (1.0% (w/v)) in 50 mmol phosphate buffer pH 8.0 was added as continuous phase during agitation at 2500 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 1.

The lyophilisate, resuspended in water contained microparticles with active principle content of 29.2% and with an encapsulations efficiency of 99.9%. The microparticles had a mean diameter of 72.6 μm.

### Example 13

1.5 g of Polymer Resomer® RG755S and 1.5 g of Polymer Resomer® RG753H were dissolved in 10 ml ethyl acetate and transferred to a double-walled glass vessel (inside height 16.0 cm, inside diameter 4.6 cm). The API solution containing 2.5 g asenapine maleate in 4.0 ml benzyl alcohol was dispersed in the polymer solution by means of a mechanical agitator (Dispermat FT, VMA-Getzmann GmbH, Germany, equipped with a 3.0 cm dissolver disc) for 15 min at 2500 rpm and for 5 min at 3500 rpm at room temperature.

80 mL PVA solution (1.0% (w/v)) in 50 mmol phosphate buffer pH 8.0 was added as continuous phase during agitation at 2500 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 1.

The lyophilisate, resuspended in water contained microparticles with active principle content of 32.1% and with an encapsulations efficiency of 98.2%. The microparticles had a mean diameter of 67.5 μm.

### Example 14

1.5 g of Polymer Resomer® RG755S and 1.5 g of Polymer Resomer® RG753H were dissolved in 10 ml ethyl formate and transferred to a double-walled glass vessel (inside height 16.0 cm, inside diameter 4.6 cm). The API solution containing 2.5 g asenapine maleate in 4.0 ml benzyl alcohol was dispersed in the polymer solution by means of a mechanical agitator (Dispermat FT, VMA-Getzmann GmbH, Germany, equipped with a 3.0 cm dissolver disc) for 5 min at 2500 rpm and for 6 min at 3500 rpm at room temperature.

80 mL PVA solution (1.0% (w/v)) in 50 mmol phosphate buffer pH 8.0 was added as continuous phase during agitation at 2500 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 1.

The lyophilisate, resuspended in water contained microparticles with active principle content of 30.4% and with an encapsulations efficiency of 92.8%. The microparticles had a mean diameter of 54.5 μm.

### Example 15

2.4 g of Polymer Resomer® RG858S and 0.6 g of Polymer Resomer® RG752H were dissolved in 15 ml ethyl acetate and transferred to a double-walled glass vessel (inside height 16.0 cm, inside diameter 4.6 cm). The API solution containing 2.0 g asenapine maleate in 4.0 ml benzyl alcohol was dispersed in the polymer solution by means of a mechanical agitator (Dispermat FT, VMA-Getzmann GmbH, Germany, equipped with a 3.0 cm dissolver disc) for 12 min at 3000 rpm and for 0.5 min at 2000 rpm at room temperature.

80 mL PVA solution (0.75% (w/v)) in 50 mmol phosphate buffer pH 8.0 was added as continuous phase during agitation at 2000 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 5.

The lyophilisate, resuspended in water contained microparticles with active principle content of 26.6% and with an encapsulations efficiency of 87.7%. The microparticles had a mean diameter of 88.9 μm.

The in-vitro release profile was measured as described for Example 1 (FIG. 8).

### Example 16

3.0 g of Polymer Resomer® RG504H was dissolved in 10 mL ethyl formate and transferred to a double-walled glass vessel (inside height 16.0 cm, inside diameter 4.6 cm). The API solution containing 2.5 g asenapine maleate in 4.0 mL benzyl alcohol was dispersed in the polymer solution by means of a mechanical agitator (Dispermat FT, VMA-Getzmann GmbH, Germany, equipped with a 3.0 cm dissolver disc) for 5 min at 3000 rpm and for 0.25 min at 2000 rpm at room temperature.

80 mL PVA solution (1% (w/v)) in 50 mmol phosphate buffer pH 8.0 was added as continuous phase during agitation at 2000 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 4.

The lyophilisate, resuspended in water contained microparticles with active principle content of 28.1% and with an encapsulations efficiency of 95.3%. The microparticles had a mean diameter of 57.3 μm.

### Example 17

1.5 g of Polymer Resomer® RG756S and 1.5 g RG753H were dissolved in 10 mL ethyl formate and transferred to a double-walled glass vessel (inside height 16.0 cm, inside diameter 4.6 cm). The API solution containing 2.5 g asenapine maleate in 4.0 mL benzyl alcohol was dispersed in the polymer solution by means of a mechanical agitator (Dispermat FT, VMA-Getzmann GmbH, Germany, equipped with a 3.0 cm dissolver disc) for 9.5 min at 3000 rpm and for 0.5 min at 2500 rpm at room temperature.

80 mL PVA solution (1% (w/v)) in 50 mmol phosphate buffer pH 8.0 was added as continuous phase during agitation at 2000 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 5.

The lyophilisate, resuspended in water contained microparticles with active principle content of 32.0% and with an encapsulations efficiency of 96.4%. The microparticles had a mean diameter of 79.3 μm.

### Example 18

1.5 g of Polymer Resomer® RG756S and 1.5 g RG753H were dissolved in 10 mL methyl acetate and transferred to a double-walled glass vessel (inside height 16.0 cm, inside diameter 3.4 cm). The API solution containing 700 mg asenapine maleate in 1.5 mL benzyl alcohol was dispersed in the polymer solution by means of a mechanical agitator (Dispermat FT, VMA-Getzmann GmbH, Germany, equipped with a 2.5 cm dissolver disc) for 10 min at 3000 rpm and for 5 min at 4000 rpm and for 0.5 min at 2400 rpm at room temperature.

80 mL PVA solution (1% (w/v)) in 50 mmol phosphate buffer pH 8.0 containing 5% (w/v) NaCl was added as continuous phase during agitation at 2000 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 5.

The lyophilisate, resuspended in water contained microparticles with active principle content of 10.6% and with an encapsulations efficiency of 76.1%. The microparticles had a mean diameter of 41.9 μm.

### Example 19

1.5 g of Polymer Resomer® RG756S and 1.5 g RG753H were dissolved in 10 mL methyl acetate and transferred to a double-walled glass vessel (inside height 16.0 cm, inside diameter 4.6 cm). The API solution containing 2.5 g asenapine maleate in 4.0 mL benzyl alcohol was dispersed in the polymer solution by means of a mechanical agitator (Dispermat FT, VMA-Getzmann GmbH, Germany, equipped with a 3.0 cm dissolver disc) for 8.5 min at 3000 rpm and for 0.25 min at 2000 rpm at room temperature.

80 mL PVA solution (1% (w/v)) in 50 mmol phosphate buffer pH 8.0 containing 5% (w/v) NaCl was added as continuous phase during agitation at 2000 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 5.

The lyophilisate, resuspended in water contained microparticles with active principle content of 24.8% and with an encapsulations efficiency of 74.6%. The microparticles had a mean diameter of 84.4 μm.

### Example 20

1.5 g of Polymer Resomer® RG755S and 1.5 g RG753H were dissolved in 10 mL ethyl formate and transferred to a double-walled glass vessel (inside height 16.0 cm, inside diameter 3.4 cm). The API solution containing 600 mg asenapine maleate in 1.5 mL benzyl alcohol was dispersed in the polymer solution by means of a mechanical agitator (Dispermat FT, VMA-Getzmann GmbH, Germany, equipped with a 2.5 cm dissolver disc) for 10 min at 3000 rpm and for 6.5 min at 4000 rpm and for 0.5 min at 3000 rpm at room temperature.

80 mL PVA solution (1.0% (w/v)) in 50 mmol phosphate buffer pH 8.0 was added as continuous phase during agitation at 3000 rpm.

After about 60 seconds of agitation, the suspension of microparticles was transferred to a 3 L beaker and 1 L PVA solution (1% (w/v)) in 50 mmol phosphate buffer pH 8.0 was added. The suspension was stirred magnetically. After 60 min another volume of 300 mL PVA solution was added. After 30 min a mixture of 100 mL PVA solution (1% (w/v)) in 50 mmol phosphate buffer pH 8.0 and 100 mL ethanol was transferred to the beaker. This was repeated twice. Subsequently, 100 mL ethanol was added after 30 min and another 100 mL after 60 min. The organic solvents ethyl acetate and benzyl alcohol were removed by extraction.

After 4 hours microparticles were collected by means of a 25 μm sieve. Subsequently, microparticles were diluted by addition of 1 L poloxamer 188 solution (4% (w/v)) in 50 mmol phosphate pH 8.0. Finally the microparticles were separated by filtration and concentrated to the desired volume. The suspension was stored frozen until lyophilisation.

The lyophilisate, resuspended in water contained microparticles with active principle content of 10.3% and with an encapsulations efficiency of 86.5%. The microparticles had a mean diameter of 81.8 μm.

### Example 21

1.5 g of Polymer Resomer® RG755S and 1.5 g RG753H were dissolved in 10 mL ethyl formate and transferred to a double-walled glass vessel (inside height 16.0 cm, inside diameter 4.6 cm). The API solution containing 2.5 g asenapine maleate in 4.0 mL benzyl alcohol was dispersed in the polymer solution by means of a mechanical agitator (Dispermat FT, VMA-Getzmann GmbH, Germany, equipped with a 3.0 cm dissolver disc) for 5 min at 2500 rpm and for 5.5 min at 3000 rpm and for 0.5 min at 2500 rpm at room temperature.

80 mL PVA solution (1.0% (w/v)) in 50 mmol phosphate buffer pH 8.0 was added as continuous phase during agitation at 2500 rpm.

After about 60 seconds of agitation, the suspension of microparticles was transferred to a 3 L beaker and 1 L PVA solution (1% (w/v)) in 50 mmol phosphate buffer pH 8.0 was added. The suspension was stirred magnetically. After 60 min another volume of 300 mL PVA solution was added. After 30 min a mixture of 100 mL PVA solution (1% (w/v)) in 50 mmol phosphate buffer pH 8.0 and 100 mL ethanol was transferred to the beaker. This was repeated twice. Subsequently, 100 mL ethanol was added after 30 min and another 100 mL after 60 min. The organic solvents ethyl acetate and benzyl alcohol were removed by extraction.

After 4 hours microparticles were collected by means of a 25 μm sieve. Subsequently, microparticles were diluted by addition of 1 L poloxamer 188 solution (4% (w/v)) in 50 mmol phosphate pH 8.0. Finally the microparticles were separated by filtration and concentrated to the desired volume. The suspension was stored frozen until lyophilisation.

The lyophilisate, resuspended in water contained microparticles with active principle content of 32.5% and with an encapsulations efficiency of 97.7%. The microparticles had a mean diameter of 16.5 μm.

### Example 22

1.5 g of Polymer Resomer® RG755S and 1.5 g of Polymer Resomer® RG753H were dissolved in 10 mL ethyl formate and transferred to a double-walled glass vessel (inside height 16.0 cm, inside diameter 4.6 cm). The API solution containing 2500 mg asenapine maleate in 2.5 mL benzyl alcohol was dispersed in the polymer solution by means of a mechanical agitator (Dispermat FT, VMA-Getzmann GmbH, Germany, equipped with a 3.0 cm dissolver disc) for 5 min at 2500 rpm and for 5.5 min at 3000 rpm and for 0.5 min at 2500 rpm at room temperature.

80 mL PVA solution (1.0% (w/v)) in 50 mmol phosphate buffer pH 8.0 was added as continuous phase during agitation at 2500 rpm.

After about 60 seconds of agitation, the suspension of microparticles was transferred to a 3 L beaker and 500 mL PVA solution (1% (w/v)) in 50 mmol phosphate buffer pH 8.0 was added. The suspension was stirred magnetically. After 30 min another 500 mL PVA solution (1% (w/v)) in 50 mmol phosphate buffer pH 8.0 containing 20% (v/v) ethanol was added to the suspension.

The organic solvents ethyl formate and benzyl alcohol were removed by extraction.

After 30 min microparticles were collected by filtration. Subsequently, microparticles were diluted by addition of 500 mL poloxamer 188 solution (10% (w/v)) in 50 mmol phosphate pH 8.0. Another 300 mL of that poloxamer 188 solution was added after 30 min. A mixture of 100 mL ethanol and 100 mL poloxamer 188 solution was added after 30 min and after 60 min. 100 mL ethanol was added after 30 min and after 60 min, and finally microparticles were separated by filtration and concentrated to the desired volume. The suspension was stored frozen until lyophilisation.

The lyophilisate, resuspended in water contained microparticles with active principle content of 30.3% and with an encapsulations efficiency of 90.2%. The microparticles had a mean diameter of 54.3 μm.

### Example 23

1.5 g of Polymer Resomer® RG755S and 1.5 g of Polymer Resomer® RG753H were dissolved in 10 mL ethyl formate and transferred to a double-walled glass vessel (inside height 16.0 cm, inside diameter 4.6 cm). The API solution containing 2.5 g asenapine maleate in 4.0 mL benzyl alcohol was dispersed in the polymer solution by means of a mechanical agitator (Dispermat FT, VMA-Getzmann GmbH, Germany, equipped with a 2.5 cm dissolver disc) for 5 min at 2500 rpm and for 9.5 min at 3500 rpm and for 0.5 min at 2500 rpm at room temperature.

80 mL PVA solution (1.0% (w/v)) in 50 mmol phosphate buffer pH 8.0 was added as continuous phase during agitation at 3000 rpm.

After about 60 seconds of agitation, the suspension of microparticles was transferred to a 3 L beaker and 500 mL PVA solution (1% (w/v)) in 50 mmol phosphate buffer pH 8.0 was added. Extraction of organic solvents ethyl acetate and benzyl alcohol was performed as described for Example 1.

The lyophilisate, resuspended in water contained microparticles with active principle content of 30.6% and with an encapsulations efficiency of 91.9%. The microparticles had a mean diameter of 82.3 μm.

### Example 24

1.5 g of Polymer Resomer® RG755S and 1.5 g of Polymer Resomer® RG753H were dissolved in 11 mL ethyl acetate and a dispersion of 2 g asenapine maleate in 3.0 mL glycofurol was added. The mixture was transferred to a double-walled glass vessel (inside height 16.0 cm, inside diameter 4.6 cm) and the API was dispersed in the polymer solution by means of a mechanical agitator (Dispermat FT, VMA-Getzmann GmbH, Germany, equipped with a 3.0 cm dissolver disc) for 10 min at 3000 rpm at room temperature.

50 mL PVA solution (2.0% (w/v)) in 50 mmol phosphate buffer pH 8.0 containing 30% sucrose (w/v) was added as continuous phase during agitation at 3000 rpm.

After about 60 seconds of agitation, the suspension of microparticles was transferred to a 1 L two-necked round bottom flask and 100 mL PVA solution (1% (w/v)) in 50 mmol phosphate buffer pH 8.0 was added. The suspension was stirred magnetically.

The solvent ethyl acetate was eliminated at room temperature by applying a vacuum and glycofurol was removed by extraction. After 90 min 50 mL 50 mmol phosphate buffer pH 8.0 containing 1% (w/v) PVA was added to the suspension and temperature was increased to 40° C. After 180 min another 50 mL 50 mmol phosphate buffer pH 8.0 containing 1% (w/v) PVA was added to the suspension. After 4 h the microparticles were washed and separated by centrifugation and concentrated to the desired volume. Microparticles were stored frozen until lyophilisation.

The lyophilisate, resuspended in water contained microparticles with active principle content of 19.5% and with an encapsulations efficiency of 72.2%. The microparticles had a mean diameter of 20.5 μm.

### Example 25

1.5 g of Polymer Resomer® RG755S and 1.5 g of Polymer Resomer® RG753H were dissolved in 11 mL ethyl acetate and a dispersion of 2 g asenapine maleate in 1.5 mL DMSO was added. The mixture was transferred to a double-walled glass vessel (inside height 16.0 cm, inside diameter 3.4 cm) and the API was dispersed in the polymer solution by means of a mechanical agitator (Dispermat FT, VMA-Getzmann GmbH, Germany, equipped with a 2.5 cm dissolver disc) for 10 min at 3000 rpm and for 22 min at 4000 rpm and for 7 min at 5000 rpm and for 1 min at 3000 rpm at room temperature.

50 mL PVA solution (2.0% (w/v)) in 50 mmol phosphate buffer pH 8.0 containing 30% sucrose (w/v) was added as continuous phase during agitation at 3000 rpm.

After about 60 seconds of agitation, the suspension of microparticles was transferred to a 1 L two-necked round bottom flask and 100 mL PVA solution (1% (w/v)) in 50 mmol phosphate buffer pH 8.0 was added. The suspension was stirred magnetically.

The solvent ethyl acetate was eliminated at room temperature by applying a vacuum and DMSO was removed by extraction. After 120 min temperature was increased to 40° C. and after 215 min 50 mL 50 mmol phosphate buffer pH 8.0 containing 1% (w/v) PVA was added to the suspension.

After 4.5 h the microparticles were washed and separated by centrifugation and concentrated to the desired volume. Microparticles were stored frozen until lyophilisation.

The lyophilisate, resuspended in water contained microparticles with active principle content of 21.7% and with an encapsulations efficiency of 81.5%. The microparticles had a mean diameter of 60.9 μm.

### Example 26

1.5 g of Polymer Resomer® RG755S and 1.5 g of Polymer Resomer® RG753H were dissolved in 11 mL ethyl acetate and a dispersion of 2 g asenapine maleate in 1.5 mL NMP was added. The mixture was transferred to a double-walled glass vessel (inside height 16.0 cm, inside diameter 3.4 cm) and the API was dispersed in the polymer solution by means of a mechanical agitator (Dispermat FT, VMA-Getzmann GmbH, Germany, equipped with a 2.5 cm dissolver disc) for 10 min at 3000 rpm and for 20 min at 4000 rpm and for 1 min at 2700 rpm at room temperature.

50 mL PVA solution (2.0% (w/v)) in 50 mmol phosphate buffer pH 8.0 containing 30% sucrose (w/v) was added as continuous phase during agitation at 2700 rpm.

After about 60 seconds of agitation, the suspension of microparticles was transferred to a 1 L two-necked round bottom flask and 100 mL PVA solution (1% (w/v)) in 50 mmol phosphate buffer pH 8.0 was added. The suspension was stirred magnetically.

The solvent ethyl acetate was eliminated at room temperature by applying a vacuum and NMP was removed by extraction. After 90 min 50 mL 50 mmol phosphate buffer pH 8.0 containing 1% (w/v) PVA was added to the suspension and temperature was increased to 40° C. After 180 min another 50 mL 50 mmol phosphate buffer pH 8.0 containing 1% (w/v) PVA was added to the suspension. After 4 h the microparticles were washed and separated by centrifugation and concentrated to the desired volume. Microparticles were stored frozen until lyophilisation.

The lyophilisate, resuspended in water contained microparticles with active principle content of 19.2% and with an encapsulations efficiency of 71.5%. The microparticles had a mean diameter of 62.9 μm.

### Example 27

1.5 g of Polymer Resomer® RG755S and 1.5 g of Polymer Resomer® RG753H were dissolved in 11 mL ethyl formate and a dispersion of 2 g asenapine maleate in 3 mL glycofurol was added. The mixture was transferred to a double-walled glass vessel (inside height 16.0 cm, inside diameter 4:6 cm) and the API was dispersed in the polymer solution by means of a mechanical agitator (Dispermat FT, VMA-Getzmann GmbH, Germany, equipped with a 3.0 cm dissolver disc) for 7 min at 3000 rpm at room temperature.

50 mL PVA solution (2.0% (w/v)) in 50 mmol phosphate buffer pH 8.0 containing 30% sucrose (w/v) was added as continuous phase during agitation at 3000 rpm.

After about 60 seconds of agitation, the suspension of microparticles was transferred to a 1 L two-necked round bottom flask and 100 mL PVA solution (1% (w/v)) in 50 mmol phosphate buffer pH 8.0 was added. The suspension was stirred magnetically.

The solvent ethyl formate was eliminated at room temperature by applying a vacuum and glycofurol was removed by extraction. After 110 min 50 mL 50 mmol phosphate buffer pH 8.0 containing 1% (w/v) PVA was added to the suspension and temperature was increased to 40° C. After 220 min another 50 mL 50 mmol phosphate buffer pH 8.0 containing 1% (w/v) PVA was added to the suspension.

After 4.5 h the microparticles were washed and separated by centrifugation and concentrated to the desired volume. Microparticles were stored frozen until lyophilisation.

The lyophilisate, resuspended in water contained microparticles with active principle content of 20.6% and with an encapsulations efficiency of 76.0%. The microparticles had a mean diameter of 46.6 μm.

### Example 28

1.5 g of Polymer Resomer® RG755S and 1.5 g of Polymer Resomer® RG753H were dissolved in 11 mL ethyl formate and a solution of 2 g asenapine maleate in 2.86 mL DMSO was added. The mixture was transferred to a double-walled glass vessel (inside height 16.0 cm, inside diameter 4:6 cm) and the API solution was dispersed in the polymer solution by means of a mechanical agitator (Dispermat FT, VMA-Getzmann GmbH, Germany, equipped with a 3.0 cm dissolver disc) for 11 min at 3000 rpm at room temperature.

50 mL PVA solution (2.0% (w/v)) in 50 mmol phosphate buffer pH 8.0 containing 30% sucrose (w/v) was added as continuous phase during agitation at 3000 rpm.

After about 60 seconds of agitation, the suspension of microparticles was transferred to a 1 L two-necked round bottom flask and 100 mL PVA solution (1% (w/v)) in 50 mmol phosphate buffer pH 8.0 was added. The suspension was stirred magnetically.

The solvent ethyl formate was eliminated at room temperature by applying a vacuum and DMSO was removed by extraction. After 110 min 50 mL 50 mmol phosphate buffer pH 8.0 containing 1% (w/v) PVA was added to the suspension and temperature was increased to 40° C. After 200 min and after 230 min aliquots of 50 mL 50 mmol phosphate buffer pH 8.0 containing 1% (w/v) PVA were added to the suspension. After 260 min the microparticles were washed and separated by centrifugation and concentrated to the desired volume. Microparticles were stored frozen until lyophilisation.

The lyophilisate, resuspended in water contained microparticles with active principle content of 22.3% and with an encapsulations efficiency of 78.4% The microparticles had a mean diameter of 81.6 μm.

### Example 29

1.5 g of Polymer Resomer® RG755S and 1.5 g of Polymer Resomer® RG753H were dissolved in 11 mL methyl acetate and a solution of 600 mg asenapine maleate in 1.4 mL DMSO was added. The mixture was transferred to a double-walled glass vessel (inside height 16.0 cm, inside diameter 3.4 cm) and the API solution was dispersed in the polymer solution by means of a mechanical agitator (Dispermat FT, VMA-Getzmann GmbH, Germany, equipped with a 2.5 cm dissolver disc) for 5 min at 3000 rpm and for 7 min at 4000 rpm and for 1 min at 3000 rpm at room temperature.

50 mL PVA solution (2.0% (w/v)) in 50 mmol phosphate buffer pH 8.0 containing 30% sucrose (w/v) was added as continuous phase during agitation at 3000 rpm.

After about 60 seconds of agitation, the suspension of microparticles was transferred to a 1 L two-necked round bottom flask and 100 mL PVA solution (1% (w/v)) in 50 mmol phosphate buffer pH 8.0 was added. The suspension was stirred magnetically.

The solvent methyl acetate was eliminated at room temperature by applying a vacuum and DMSO was removed by extraction. After 90 min 50 mL 50 mmol phosphate buffer pH 8.0 containing 1% (w/v) PVA was added to the suspension and temperature was increased to 40° C. After 180 min and after 210 min aliquots of 50 mL 50 mmol phosphate buffer pH 8.0 containing 1% (w/v) PVA were added to the suspension. After 240 min the microparticles were washed and separated by centrifugation and concentrated to the desired volume. Microparticles were stored frozen until lyophilisation.

The lyophilisate, resuspended in water contained microparticles with active principle content of 10.3% and with an encapsulations efficiency of 85.1%. The microparticles had a mean diameter of 41.1 μm.

### Example 30

1.5 g of Polymer Resomer® RG755S and 1.5 g of Polymer Resomer® RG753H were dissolved in 11 mL methyl acetate and a solution of 2 g asenapine maleate in 2.86 mL DMSO was added. The mixture was transferred to a double-walled glass vessel (inside height 16.0 cm, inside diameter 4:6 cm) and the API solution was dispersed in the polymer solution by means of a mechanical agitator (Dispermat FT, VMA-Getzmann GmbH, Germany, equipped with a 3.0 cm dissolver disc) for 12 min at 3000 rpm at room temperature.

50 mL PVA solution (2.0% (w/v)) in 50 mmol phosphate buffer pH 8.0 containing 30% sucrose (w/v) was added as continuous phase during agitation at 3000 rpm.

After about 60 seconds of agitation, the suspension of microparticles was transferred to a 1 L two-necked round bottom flask and 100 mL PVA solution (1% (w/v)) in 50 mmol phosphate buffer pH 8.0 was added. The suspension was stirred magnetically.

The solvent methyl acetate was eliminated at room temperature by applying a vacuum and DMSO was removed by extraction. After 90 min 50 mL 50 mmol phosphate buffer pH 8.0 containing 1% (w/v) PVA was added to the suspension and temperature was increased to 40° C. After 180 min and after 210 min aliquots of 50 mL 50 mmol phosphate buffer pH 8.0 containing 1% (w/v) PVA were added to the suspension. After 240 min the microparticles were washed and separated by centrifugation and concentrated to the desired volume. Microparticles were stored frozen until lyophilisation.

The lyophilisate, resuspended in water contained microparticles with active principle content of 23.1% and with an encapsulations efficiency of 81.3%. The microparticles had a mean diameter of 116.2 μm.

### Example 31

1.5 g of Polymer Resomer® RG755S and 1.5 g of Polymer Resomer® RG753H were dissolved in 10 mL ethyl formate and a dispersion of 1 g asenapine maleate in 1.5 mL glycofurol was added. The mixture was transferred to a double-walled glass vessel (inside height 16.0 cm, inside diameter 3.4 cm) and the API was dispersed in the polymer solution by means of a mechanical agitator (Dispermat FT, VMA-Getzmann GmbH, Germany, equipped with a 2.5 cm dissolver disc) for 6 min at 3000 rpm and for 8 min at 4000 rpm and for 1 min at 3500 rpm at room temperature.

50 mL poloxamer 188 solution (4.0% (w/v)) in 50 mmol phosphate buffer pH 8.0 containing 30% sucrose (w/v) was added as continuous phase during agitation at 3500 rpm.

After about 60 seconds of agitation, the suspension of microparticles was transferred to a 1 L two-necked round bottom flask and 100 mL poloxamer 188 solution (16% (w/v)) in 50 mmol phosphate buffer pH 8.0 was added. The suspension was stirred magnetically.

The solvent ethyl formate was eliminated at room temperature by applying a vacuum and glycofurol was removed by extraction. After 90 min 50 mL 50 mmol phosphate buffer pH 8.0 containing 16% (w/v) poloxamer 188 was added to the suspension and temperature was increased to 40° C. After 180 min and after 210 min aliquots of 50 mL 50 mmol phosphate buffer pH 8.0 containing 16% (w/v) poloxamer 188 were added to the suspension. After 240 min the microparticles were washed and separated by centrifugation and concentrated to the desired volume. Microparticles were stored frozen until lyophilisation.

The lyophilisate, resuspended in water contained microparticles with active principle content of 13.8% and with an encapsulations efficiency of 78.2%. The microparticles had a mean diameter of 29.1 μm.

### Example 32

1.5 g of Polymer Resomer® RG755S and 1.5 g of Polymer Resomer® RG753H were dissolved in 11 mL ethyl formate and a solution of 1 g asenapine maleate in 1.5 mL DMSO was added. The mixture was transferred to a double-walled glass vessel (inside height 16.0 cm, inside diameter 3.4 cm) and the API was dispersed in the polymer solution by means of a mechanical agitator (Dispermat FT, VMA-Getzmann GmbH, Germany, equipped with a 2.5 cm dissolver disc) for 7 min at 3000 rpm and for 9 min at 4000 rpm and for 0.5 min at 3500 rpm at room temperature.

50 mL poloxamer 188 solution (4.0% (w/v)) in 50 mmol phosphate buffer pH 8.0 containing 30% sucrose (w/v) was added as continuous phase during agitation at 3500 rpm.

After about 60 seconds of agitation, the suspension of microparticles was transferred to a 1 L two-necked round bottom flask and 100 mL poloxamer 188 solution (16% (w/v)) in 50 mmol phosphate buffer pH 8.0 was added. The suspension was stirred magnetically.

The solvent ethyl formate was eliminated at room temperature by applying a vacuum and DMSO was removed by extraction. After 90 min 50 mL 50 mmol phosphate buffer pH 8.0 containing 16% (w/v) poloxamer 188 was added to the suspension and temperature was increased to 40° C. After 180 min and after 210 min aliquots of 50 mL 50 mmol phosphate buffer pH 8.0 containing 16% (w/v) poloxamer 188 were added to the suspension. After 240 min the microparticles were washed and separated by centrifugation and concentrated to the desired volume. Microparticles were stored frozen until lyophilisation.

The lyophilisate, resuspended in water contained microparticles with active principle content of 14.1% and with an encapsulations efficiency of 78.0%. The microparticles had a mean diameter of 19.7 μm.

### Comparative Example 1—Double Emulsion Experiment

50 mg asenapine maleate was dissolved in 2.0 ml dichloromethane. The solution was added to a solution of 100 mg of Polymer Resomer® RG755S and 100 mg of Polymer Resomer® RG753H in 5 ml dichloromethane. The clear solution was poured into 700 mL PVA solution (0.5% wt/v) under continuous stirring at 2000 rpm by means of a mechanical agitator (Dispermat FT, VMA-Getzmann GmbH, Germany, equipped with a 4.0 cm dissolver disc) in a double-walled glass vessel (inside height 215 mm, inside diameter 110 mm). After pouring the emulsion was stirred for 3 h at 1500 rpm. Finally, microparticles were washed and collected by filtration.

The lyophilisate, resuspended in water contained microparticles with active principle content of 2.8% and with an encapsulations efficiency of 20.0%. The microparticles had a mean diameter of 105.2 μm.

### Comparative Example 2—Double Emulsion Experiment

167 mg asenapine maleate was dispersed in 5.0 ml dichloromethane. The solution was added to a solution of 100 mg of Polymer Resomer® RG755S and 100 mg of Polymer Resomer® RG753H in 7.5 ml dichloromethane. The clear solution was poured into 700 mL PVA solution (0.5% wt/v) under continuous stirring at 2000 rpm by means of a mechanical agitator (Dispermat FT, VMA-Getzmann GmbH, Germany, equipped with a 4.0 cm dissolver disc) in a double-walled glass vessel (inside height 215 mm, inside diameter 110 mm). After pouring the emulsion was stirred for 3 h at 1500 rpm. Finally, microparticles were washed and collected by filtration.

The lyophilisate, resuspended in water contained microparticles with active principle content of 1.4% and with an encapsulations efficiency of 4.4%. The microparticles had a mean diameter of 40.9 μm.

### DESCRIPTION OF THE FIGURES

FIG. 1 shows the in-vitro release profile of the composition of Example 1.

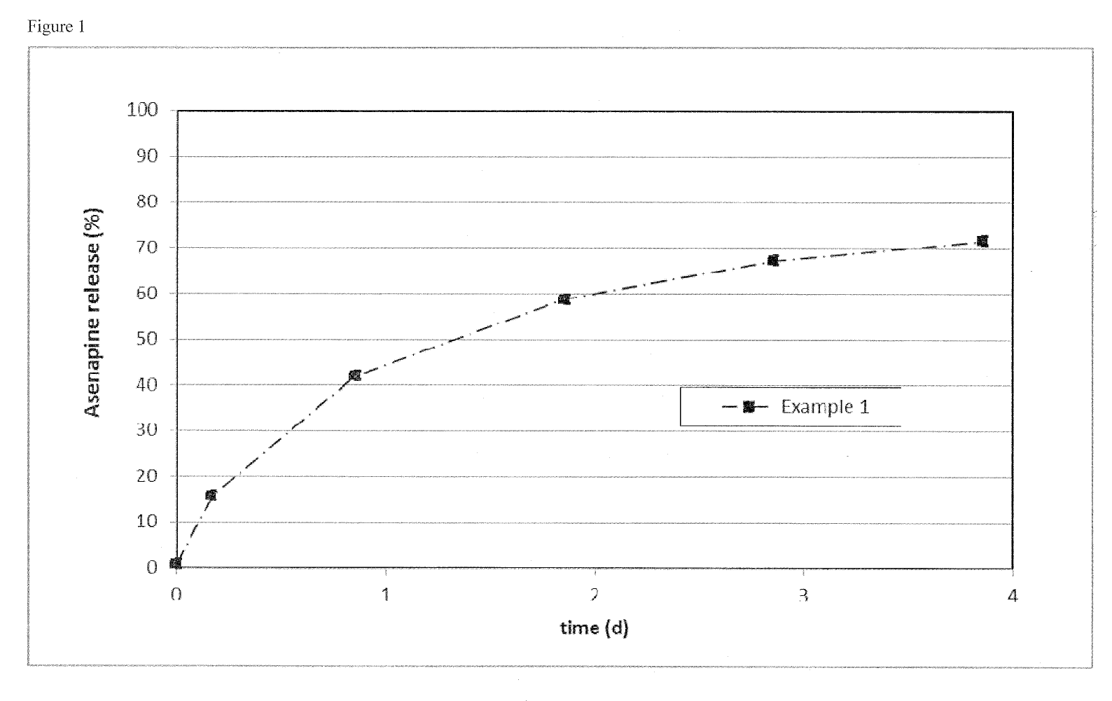

FIG. 2 shows the in-vitro release profile of the composition of Example 2.

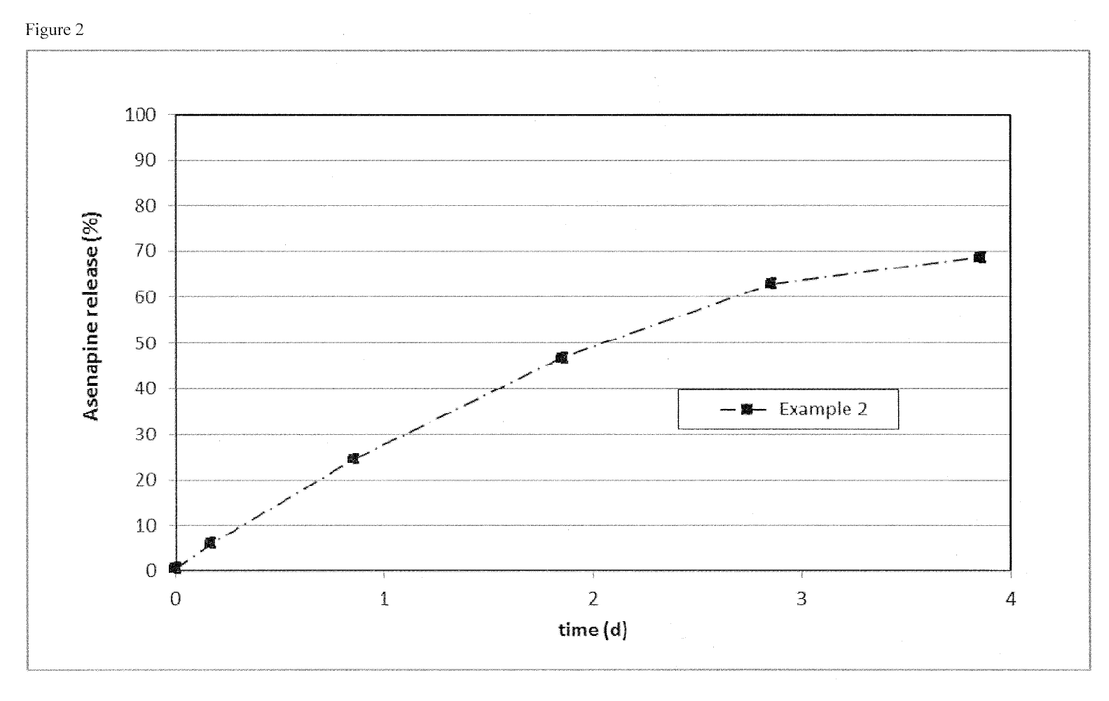

FIG. 3 shows the in-vitro release profile of the composition of Example 3

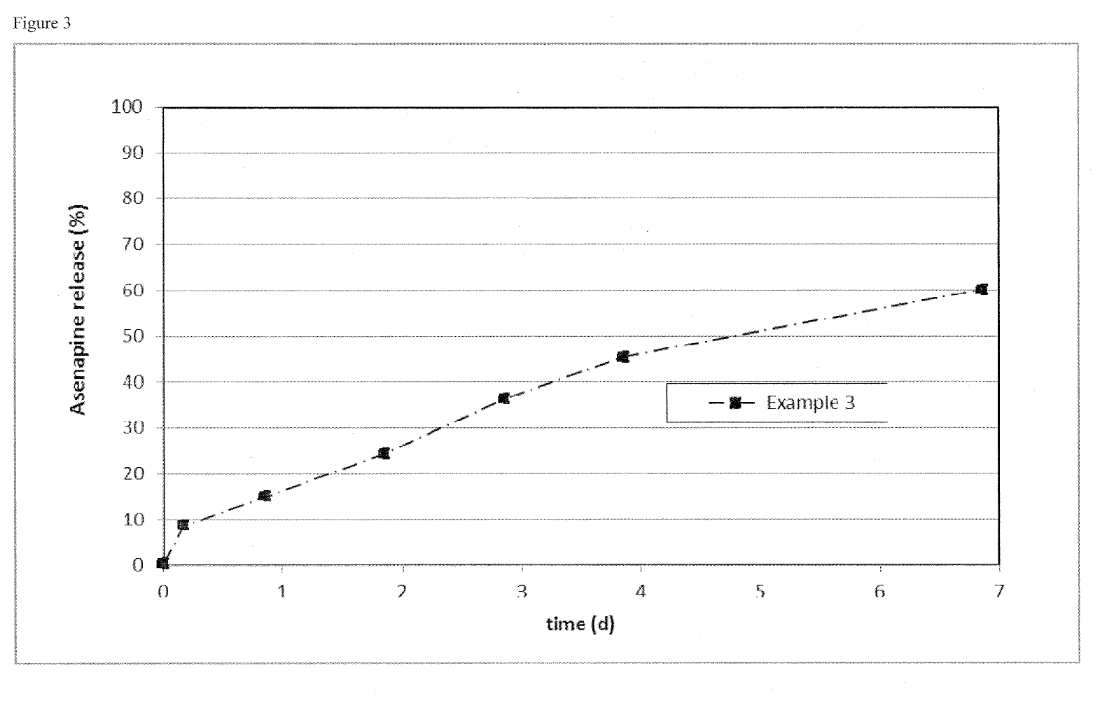

FIG. 4 shows the particle size distribution of the composition of Example 4 determined by means of laser diffraction (Beckmann Coulter LS 13 320).

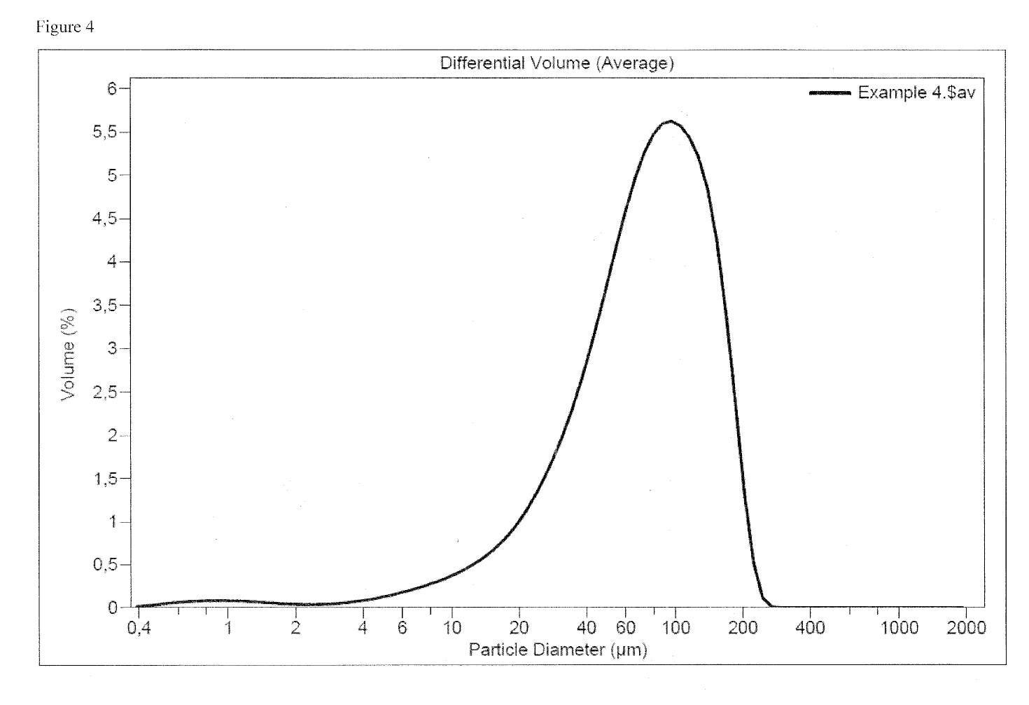

FIG. 5 shows the in-vitro release profile of the composition of Example 5.

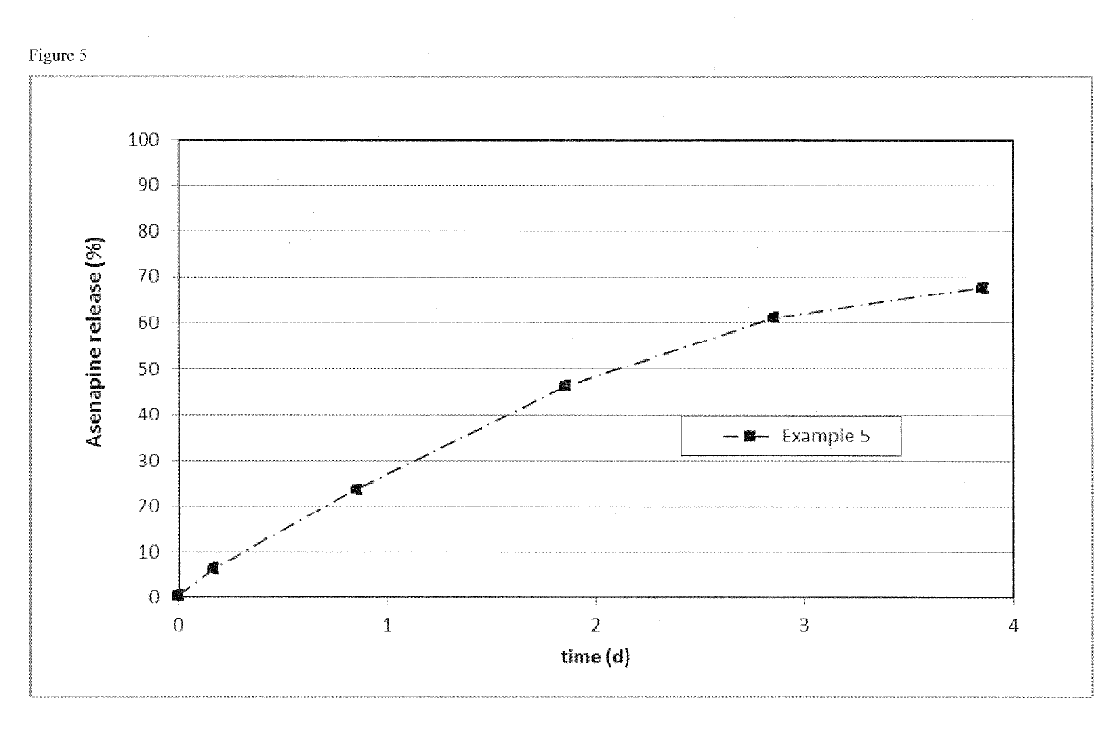

FIG. 6 shows the in-vitro release profile of the composition of Example 6.

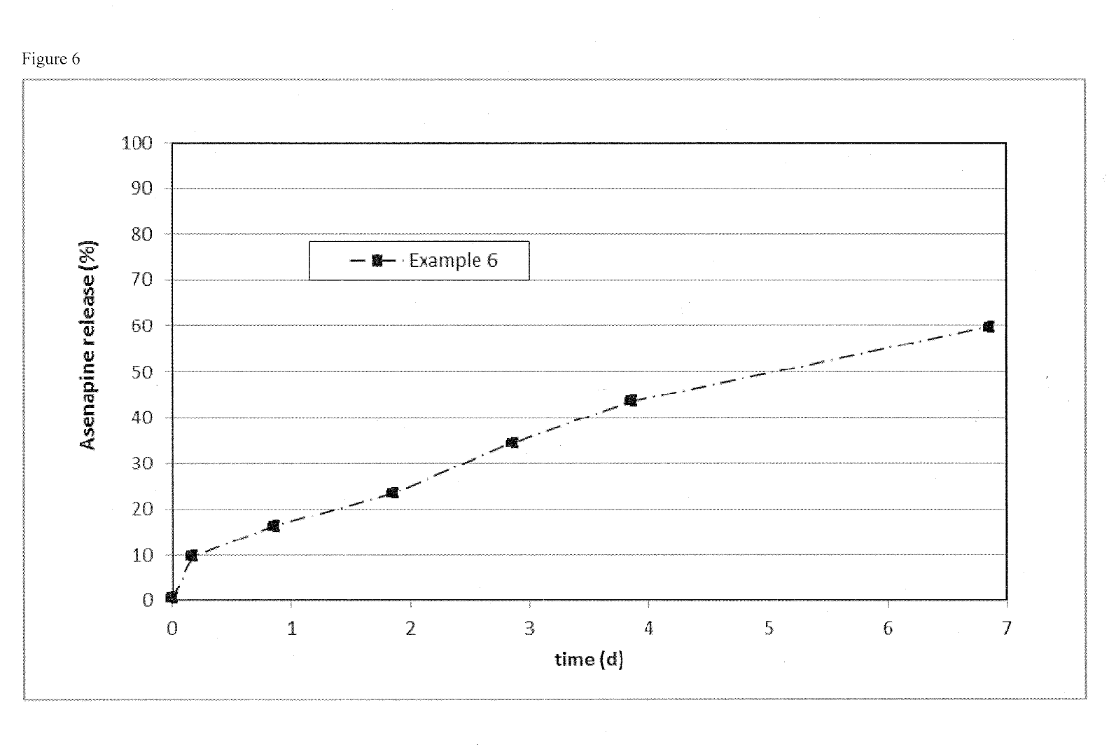

FIG. 7 shows the in-vitro release profile of the composition of Example 7.

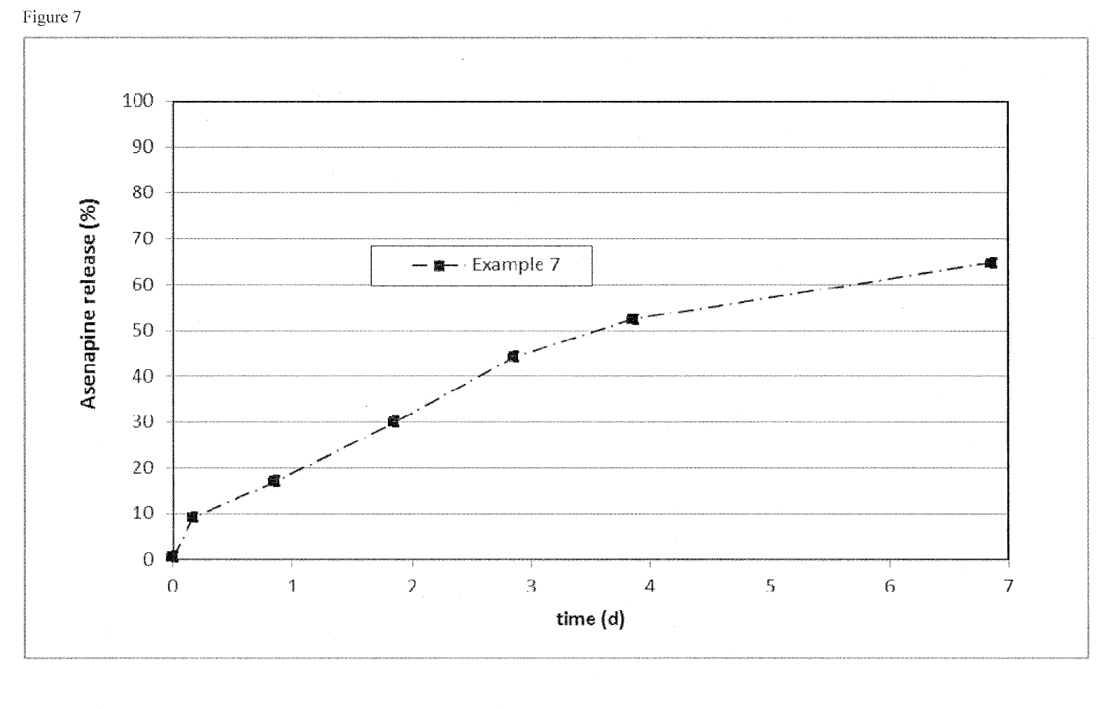

FIG. 8 shows the in-vitro release profile of the composition of Example 15.

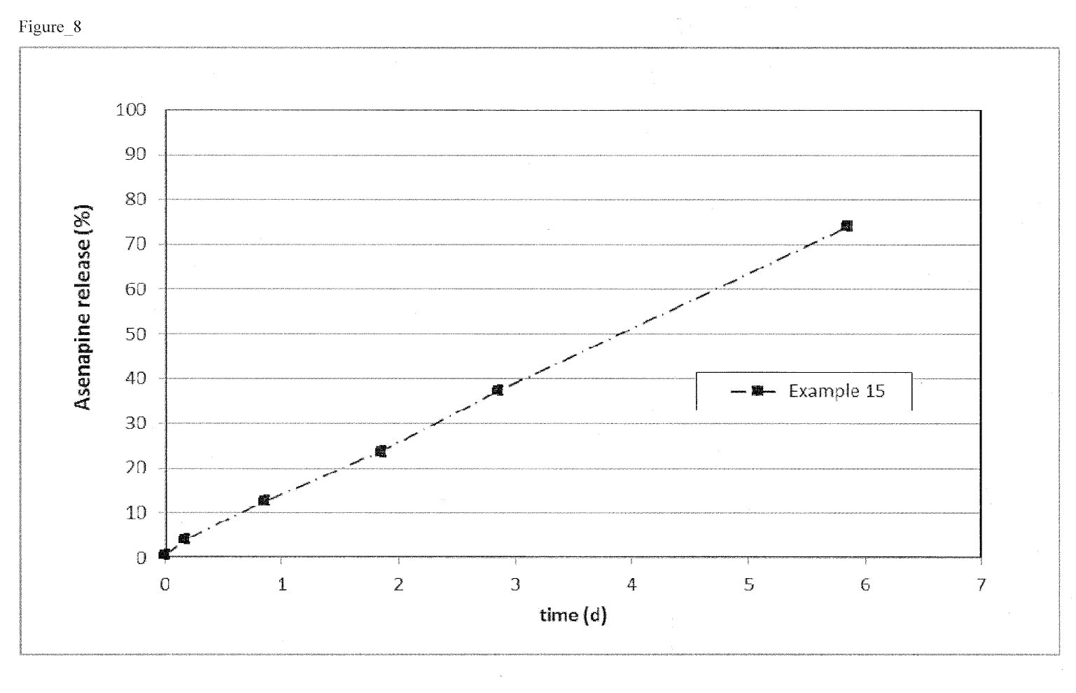

1. Nano- and/or microparticles comprising asenapine or a pharmaceutically acceptable salt thereof, wherein said asenapine or said pharmaceutically acceptable salt thereof is embedded in a polymer matrix or encapsulated by a polymer shell, wherein the polymer matrix or the polymer shell comprises a polymer selected from polylactide, polyglycolide, and polyester copolymers comprising copolymerized units of lactic acid and/or glycolic acid.

2. The nano- and/or microparticles of claim 1, which comprise the asenapine in the form of the asenapine free base, or in the form of a salt selected from hydrochloride, hydrobromide, hydroiodide, sulfate, nitrate, phosphate, carbonate, hydrogencarbonate, perchlorate, acetate, propionate, butyrate, pentanoate, hexanoate, heptanoate, octanoate, cyclopentanepropionate, undecanoate, lactate, maleate, oxalate, fumarate, tartrate, malate, citrate, nicotinate, benzoate, salicylate, pamoate, ascorbate, methanesulfonate, ethanesulfonate, 2-hydroxyethanesulfonate, benzenesulfonate, p-toluenesulfonate (tosylate), 2-naphthalenesulfonate, 3-phenylsulfonate, camphorsulfonate, aspartate and glutamate salt.

3. The nano- and/or microparticles of claim 1, which comprise the asenapine in the form of a maleate salt.

4. The nano- and/or microparticles of any of claims 1 to 3, which are obtainable by a process comprising the steps of
a) providing a solution of a polymer in an organic solvent S1 having limited water solubility; wherein said polymer is selected from the group consisting of polylactide, polyglycolide, and polyester copolymers comprising copolymerized units of lactic acid and/or glycolic acid;
b) combining the solution provided in step a) with asenapine or a pharmaceutically acceptable salt thereof by
b1) dispersing the asenapine or the pharmaceutically acceptable salt thereof in the solution provided in step a), or
b2) providing a solution or dispersion of the asenapine or the pharmaceutically acceptable salt thereof in an organic solvent S2 and combining the solution or dispersion with the solution provided in step a),
to provide an organic phase which comprises dissolved polymer and asenapine or a pharmaceutically acceptable salt thereof dissolved or dispersed therein;
c) agitating the organic phase provided in step b) in a vessel and adding an aqueous surfactant solution to the agitated organic phase in a volume ratio of at least 2:1 in terms of the total volume of the aqueous surfactant solution to the total volume of the organic phase provided in step b), thus causing the formation of a dispersion containing a continuous aqueous phase and a discontinuous organic phase; and
d) allowing the formation of a suspension of the nano- and/or microparticles via transfer of organic solvent from the discontinuous organic phase into the aqueous surfactant phase directly after the dispersion has been formed.

5. The nano- and/or microparticles of any of claims 1 to 4, wherein the content of asenapine or the pharmaceutically acceptable salt thereof is at least 10 wt. %, based on the total weight of the particles.

6. The nano- and/or microparticles of any of claims 1 to 4, wherein the content of asenapine or the pharmaceutically acceptable salt thereof is at least 20 wt. %, based on the total weight of the particles.

7. The nano- and/or microparticles of any of claims 1 to 6, wherein the asenapine or the pharmaceutically acceptable salt thereof is dispersed as an amorphous or crystalline solid in a polymer matrix.

8. The nano- and/or microparticles of any of claims 1 to 7, wherein the polymer matrix or polymer shell comprises a poly(lactide-co-glycolide) copolymer.

9. The nano- and/or microparticles of any of claims 1 to 8, which have a volume weighted mean particle size of 1 μm to 125 μm.

10. A process for the production of the nano- and/or microparticles of any of claims 1 to 9, said process comprising the steps of
a) providing a solution of a polymer in an organic solvent S1 having limited water solubility; wherein said polymer is selected from the group consisting of polylactide, polyglycolide, and polyester copolymers comprising copolymerized units of lactic acid and/or glycolic acid;
b) combining the solution provided in step a) with asenapine or a pharmaceutically acceptable salt thereof by
b1) dispersing the asenapine or the pharmaceutically acceptable salt thereof in the solution provided in step a), or
b2) providing a solution or dispersion of the asenapine or the pharmaceutically acceptable salt thereof in an organic solvent S2 and combining the solution or dispersion with the solution provided in step a),
to provide an organic phase which comprises dissolved polymer and asenapine or a pharmaceutically acceptable salt thereof dissolved or dispersed therein;
c) agitating the organic phase provided in step b) in a vessel and adding an aqueous surfactant solution to the agitated organic phase in a volume ratio of at least 2:1 in terms of the total volume of the aqueous surfactant solution to the total volume of the organic phase provided in step b), thus causing the formation of a dispersion containing a continuous aqueous phase and a discontinuous organic phase; and
d) allowing the formation of a suspension of the nano- and/or microparticles via transfer of organic solvent from the discontinuous organic phase into the aqueous surfactant phase directly after the dispersion has been formed.

11. The process of claim 10, wherein the solvent S1 is selected from alkyl acetates, alkyl formates, and mixtures of two or more thereof.

12. The process of claim 10 or 11, wherein the solution provided in step a) is combined with asenapine or a pharmaceutically acceptable salt thereof by b2) providing a solution or dispersion of the asenapine or the pharmaceutically acceptable salt thereof in an organic solvent S2 and combining the solution or dispersion with the solution provided in step a).

13. The process of claim 12, wherein the organic solvent S2 is selected from alkyl esters of benzoic acid, aryl esters of benzoic acid, benzyl alcohol, dimethyl sulfoxide, N-methyl pyrrolidone, glycofurol and mixtures thereof.

14. A pharmaceutical formulation comprising the nano- and/or microparticles of any of claims 1 to 9, optionally in combination with a pharmaceutically acceptable excipient.

15. The pharmaceutical formulation of claim 14 for use in the treatment or prevention of a mental disorder selected from schizophrenia, bipolar disorder, and symptoms associated with schizophrenia or bipolar disorder.

16. The pharmaceutical formulation of claim 14 or 15, which is to be administered via subcutaneous or intramuscular injection.

17. The pharmaceutical formulation of any of claims 14 to 16, which is to be administered in intervals of at least 1 week between consecutive administrations.

18. A kit containing, in separate containers or compartments, (i) the nano- and/or microparticles according to any of claims 1 to 9 in the form of a dry powder and (ii) water for injection or a physiological solution for reconstitution of the powder.
